SEQUENCE LISTING (11 sequences)

SEQ ID NO: 1 - DNA, 348 bp, Artificial sequence
  Engineered chimeric rabbit/human sequence
gagcagctggtggagtccgggggtcgcctggtcacgcctgggacagccctgacactcacctgcaaagtctctggattctccctcagtggcttctggctgaactgggtccgccaggctccagggaaggggctggagtgggtcggagccatttacagaggtagtggtagtgaatggtacgcgagctgggcaaaaggccgattcactatctccgatacctcgaccacggtgactctgaaactgaccagtccaacaaccgaggacacggccacctatttctgtgccgctgatactactgataatgggtactttaccatctggggcccaggcaccctggtcaccgtctcctca

SEQ ID NO: 2 - protein, 225 aa, Artificial sequence
  Engineered chimeric rabbit/human sequence
QEQLVESGGRLVTPGTALTLTCKVSGFSLSGFWLNWVRQAPGKGLEWVGAIYRGSGSEWYASWAKGRFTISDTSTTVTLKLTSPTTEDTATYFCAADTTDNGYFTIWGPGTLVTVSSASTKGPSVFPLAPSAKSTSGGTAALGCLVKDYFPEPVTVSWNSGALTSGVHTFPAVLQSSGLYSLSSVVTVPSSSLGTQTYICNVNHKPSNTKVDKKAEPKSCDKTRG

SEQ ID NO: 3 - DNA, 333 bp, Artificial sequence
  Engineered chimeric rabbit/human sequence
gccgagctcgtgatgacccagactccatcctccgtgtctgaacctgtgggaggcacagtcaccatcaagtgccaggccagtcagagcattagcagttggttatcctggtatcagcagaaaccagggcagcctcccaagctcctgatctacgatgcatccaatctggcatctggggtcccgtcgcgatttatgggcagtgggtctgggacagagtacactctcaccatcagcggcgtgcagcgtgaggatgctgccacctactactgtctaggtggttatcctgctgcttcttatcgaactgctttcggcggagggaccgagctggagatcata

SEQ ID NO: 4 - protein, 218 aa, Artificial sequence
  Engineered chimeric rabbit/human sequence
AELVMTQTPSSVSEPVGGTVTIKCQASQSISSWLSWYQQKPGQPPKLLIYDASNLASGVPSRFMGSGSGTEYTLTISGVQREDAATYYCLGGYPAASYRTAFGGGTELEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1512 bp, Artificial sequence
  Engineered chimeric rabbit/human sequence
atgaaaaagacagctatcgcgattgcagtggcactggctggtttcgctaccgtggcccaggcggccgagctcgtgatgacccagactccatcctccgtgtctgaacctgtgggaggcacagtcaccatcaagtgccaggccagtcagagcattagcagttggttatcctggtatcagcagaaaccagggcagcctcccaagctcctgatctacgatgcatccaatctggcatctggggtcccgtcgcgatttatgggcagtgggtctgggacagagtacactctcaccatcagcggcgtgcagcgtgaggatgctgccacctactactgtctaggtggttatcctgctgcttcttatcgaactgctttcggcggagggaccgagctggagatcatacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttaattctagataattaattaggaggaatttaaaatgaaatacctattgcctacggcagccgctggattgttattactcgctgcccaaccagccatggcccaggagcagctggtggagtccgggggtcgcctggtcacgcctgggacagccctgacactcacctgcaaagtctctggattctccctcagtggcttctggctgaactgggtccgccaggctccagggaaggggctggagtgggtcggagccatttacagaggtagtggtagtgaatggtacgcgagctgggcaaaaggccgattcactatctccgatacctcgaccacggtgactctgaaactgaccagtccaacaaccgaggacacggccacctatttctgtgccgctgatactactgataatgggtactttaccatctggggcccaggcaccctggtcaccgtctcctcagcctccaccaagggcccatcggtcttccccttggccccgtcggccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactaggggtcatcatcaccatcaccattga

SEQ ID NO: 6 - protein, 239 aa, Artificial sequence
  Engineered chimeric rabbit/human sequence
MKKTAIAIAVALAGFATVAQAAELVMTQTPSSVSEPVGGTVTIKCQASQSISSWLSWYQQKPGQPPKLLIYDASNLASGVPSRFMGSGSGTEYTLTISGVQREDAATYYCLGGYPAASYRTAFGGGTELEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 253 aa, Artificial sequence
  Engineered chimeric rabbit/human sequence
MKYLLPTAAAGLLLLAAQPAMAQEQLVESGGRLVTPGTALTLTCKVSGFSLSGFWLNWVRQAPGKGLEWVGAIYRGSGSEWYASWAKGRFTISDTSTTVTLKLTSPTTEDTATYFCAADTTDNGYFTIWGPGTLVTVSSASTKGPSVFPLAPSAKSTSGGTAALGCLVKDYFPEPVTVSWNSGALTSGVHTFPAVLQSSGLYSLSSVVTVPSSSLGTQTYICNVNHKPSNTKVDKKAEPKSCDKTRGHHHHHH

SEQ ID NO: 8 - DNA, 822 bp, Artificial sequence
  Engineered chimeric rabbit/human sequence
atgaaaaagacagctatcgcgattgcagtggcactggctggtttcgctaccgtggcccaggcggccgagctcgtgatgacccagactccatcctccgtgtctgaacctgtgggaggcacagtcaccatcaagtgccaggccagtcagagcattagcagttggttatcctggtatcagcagaaaccagggcagcctcccaagctcctgatctacgatgcatccaatctggcatctggggtcccgtcgcgatttatgggcagtgggtctgggacagagtacactctcaccatcagcggcgtgcagcgtgaggatgctgccacctactactgtctaggtggttatcctgctgcttcttatcgaactgctttcggcggagggaccgagctggagatcataggtggctcctctcgctcgagctcttccggtggcggtggctctggcggtggcggtcaggagcagctggtggagtccgggggtcgcctggtcacgcctgggacagccctgacactcacctgcaaagtctctggattctccctcagtggcttctggctgaactgggtccgccaggctccagggaaggggctggagtgggtcggagccatttacagaggtagtggtagtgaatggtacgcgagctgggcaaaaggccgattcactatctccgatacctcgaccacggtgactctgaaactgaccagtccaacaaccgaggacacggccacctatttctgtgccgctgatactactgataatgggtactttaccatctggggcccaggcaccctggtcaccgtctcctcacatcaccatcaccatcactag

SEQ ID NO: 9 - protein, 273 aa, Artificial sequence
  Engineered chimeric rabbit/human sequence
MKKTAIAIAVALAGFATVAQAAELVMTQTPSSVSEPVGGTVTIKCQASQSISSWLSWYQQKPGQPPKLLIYDASNLASGVPSRFMGSGSGTEYTLTISGVQREDAATYYCLGGYPAASYRTAFGGGTELEIIGGSSRSSSSGGGGSGGGGQEQLVESGGRLVTPGTALTLTCKVSGFSLSGFWLNWVRQAPGKGLEWVGAIYRGSGSEWYASWAKGRFTISDTSTTVTLKLTSPTTEDTATYFCAADTTDNGYFTIWGPGTLVTVSSHHHHHH

SEQ ID NO: 10 - protein, 252 aa, Artificial sequence
  Engineered chimeric rabbit/human sequence
AELVMTQTPSSVSEPVGGTVTIKCQASQSISSWLSWYQQKPGQPPKLLIYDASNLASGVPSRFMGSGSGTEYTLTISGVQREDAATYYCLGGYPAASYRTAFGGGTELEIIGGSSRSSSSGGGGSGGGGQEQLVESGGRLVTPGTALTLTCKVSGFSLSGFWLNWVRQAPGKGLEWVGAIYRGSGSEWYASWAKGRFTISDTSTTVTLKLTSPTTEDTATYFCAADTTDNGYFTIWGPGTLVTVSSHHHHHH

SEQ ID NO: 11 - protein, 9 aa, artificial sequence
  Engineered Peptide
RKKRRQRRR

(19) United States
(12) Patent Application Publication
(10) Pub. No.: US 2012/0201811 A1
(43) Pub. Date: Aug. 9, 2012

(54) GENERATION AND USE OF FAB, SCFV, AND RELATED BINDING MOLECULES SPECIFIC FOR HIV-1 REV

(75) Inventors: Stephen J. Stahl, Bethesda, MD (US); Norman R. Watts, Dickerson, MD (US); Paul T. Wingfield, Bethesda, MD (US); Christoph Rader, Olney, MD (US); Alasdair C. Steven, Silver Spring, MD (US)

(73) Assignees: Secretary, Department of Health and Human Services; The United States of America as represented by the

(21) Appl. No.: 13/366,015

(22) Filed: Feb. 3, 2012

Related U.S. Application Data
(60) Provisional application No. 61/439,307, filed on Feb. 3, 2011.

(51) Int. Cl. A61K 39/395; C12N 15/13; C12N 15/63; C12N 1/21; A61P 31/18; C12N 1/15; C12N 5/10; C12N 9/96; C12N 7/06; C07K 2/00; C07K 16/10; C12N 1/19

(52) U.S. Cl. 424/133.1; 530/387.3; 536/23.53; 435/320.1; 435/252.33; 435/252.34; 435/252.31; 435/254.2; 435/254.11; 435/328; 435/419; 530/391.3; 530/391.7; 435/188; 530/391.1; 435/238; 530/300

(57) ABSTRACT

Described herein is the identification, though phage display, of a chimeric rabbit/human anti-Rev Fab (SJS-R1) that readily solubilized polymeric HIV-1 Rev. The Fab binds with very high affinity to a conformational epitope in the N-terminal half of HIV-1 Rev. The corresponding single chain antibody (scFv) was also prepared and characterized. Methods of making and using SJS-R1 Fab and SJS-R1 scFv, and antibodies and antibody fragments that share at least one CDR with SJS-R1 Fab, are provided. Specific described methods include methods of preventing or reversing polymerization of HIV Rev, methods of preventing or inhibiting replication of a lentivirus in a cell, methods of reducing infectivity of replication of a lentivirus, inhibiting Rev function in a cell infected with a lentivirus, and methods of treating a disease or symptom associated with Rev expression in an animal.

### CROSS REFERENCE TO RELATED APPLICATION

This application claims the benefit of the earlier filing date of U.S. Provisional Application No. 61/439,307, filed Feb. 3, 2011, the entire content of which is incorporated herein by reference.

### FIELD

The present disclosure relates to methods and compositions for treating and/or inhibiting viral infection, particularly the treatment and/or inhibition of infection by a lentivirus such as HIV.

### BACKGROUND

The unique antibody repertoire development in rabbits (Oryctolagus cuniculus) has been exploited for the generation of polyclonal and monoclonal antibodies with exceptionally high avidity, affinity, and specificity (Mage et al., Dev Comp Immunol 30, 137-53, 2006). While rabbit polyclonal antibodies have had a long standing as research reagents, the more recent generation of rabbit monoclonal antibodies through both phage display (Ridder et al., Biotechnology 13, 255-60, 1995; Lang et al., Gene 172, 295-8, 1996; Foti et al., J Immunol Methods 213, 201-12, 1998; Rader et al., J Biol Chem 275, 13668-76, 2000) and hybridoma technology (Spieker-Polet et al., Proc Natl Acad Sci USA 92, 9348-52, 1995) has provided access to a highly defined research reagent of unlimited supply. In addition to high affinity and specificity, rabbit monoclonal antibodies can recognize epitopes conserved between human, mouse, and rat antigens (Popkov et al., J Immunol Methods 288, 149-64, 2004; Popkov et al., J Mol Biol 325, 325-35, 2003; Hofer et al., J Immunol Methods 318, 75-87, 2007). This cross-reactivity along with the demonstration that rabbit monoclonal antibodies can be humanized has raised an interest in utilizing rabbit monoclonal antibodies for therapeutic, applications (Rader, Drug Discov Today 6, 36-43, 2001). Their ability to target epitopes that differ from those recognized by mouse monoclonal antibodies makes rabbit monoclonal antibodies attractive research reagents for functional and biophysical studies of antigen/antibody interactions.

A rabbit monoclonal antibody format of particular interest is the chimeric rabbit/human Fab which consist of rabbit variable domains V_{H} and V_{K} and human constant domains C_{γ1}1 and C_{κ} (Rader et al., J Biol Chem 275, 13668-76, 2000; Rader, Methods Mol Biol 525, 101-28, xiv. 2009). Chimeric rabbit/human Fab libraries can be generated from spleen and bone marrow of immunized rabbits, in particular b9 allotype rabbits (Popkov et al., J Immunol Methods 288, 149-64, 2004; Popkov et al., J Mol Biol 325, 325-35, 2003; Hofer et al., J Immunol Methods 318, 75-87, 2007), and subsequently selected by phage display to yield chimeric rabbit/human Fab of high affinity, specificity, cross-reactivity, and convertibility to chimeric rabbit/human IgG1. The rabbit variable domains V_{H} and V_{κ} of chimeric rabbit/human Fab can be humanized (Rader et al., J Biol Chem 275, 13668-76, 2000; Steinberger et al., J Biol Chem 275, 36073-8, 2000).

While the 150-kDa IgG molecule is the most commonly used format of monoclonal antibodies in basic research as well as diagnostic, preventative, and therapeutic applications, the smaller 50-kDa Fab molecule, which can be expressed in E. coli, has facilitated the generation, affinity maturation, and humanization of monoclonal antibodies through in vitro evolution technologies, most prominently phage display (Rader, Drug Discov Today 6, 36-43, 2001; Rader & Barbas, Curr Opin Biotechnol 8, 503-8, 1997; Hoogenboom, Nat Biotechnol 23, 1105-16, 2005). Thus, in most instances, the Fab molecule has been an enabling format for generating and evolving IgG for particular purposes. Nonetheless, Fab have also been utilized in their own right for an increasing number of applications that exploit its smaller size and easier manufacturability compared to IgG (Rader, Curr Protoc Protein Sci Chapter 6, Unit 6 9, 2009).

An important application is the utilization of Fab for the co-crystallization of proteins in general and transmembrane, hydrophobic, and aggregating proteins in particular. In addition to providing crystal contacts through protruding hydrophilic surfaces, Fab can support crystallization by locking in conformations and blocking aggregation. For example, Fab have been used as crystal chaperones (Ye et al., Proc Nall Acad Sci USA 105, 82-7, 2008) in the determination of the three-dimensional structure of transmembrane ion channels and G protein-coupled receptors (Zhou et al., Nature 414, 43-8, 2001; Rasmussen et al., Nature 450, 383-7, 2007; Uysal et al., Proc Natl Acad Sci USA 106, 6644-9, 2009). Notably, phage display has facilitated the generation and evolution of Fab with superior co-crystallization properties (Ye et al., Proc Nall Acad Sci USA 105, 82-7, 2008; Uysal et al., Proc Natl Acad Sci USA 106, 6644-9, 2009).

Acquired immune deficiency syndrome or acquired immunodeficiency syndrome (AIDS) is a disease of the human immune system caused by the human immunodeficiency virus (HIV). This condition progressively reduces the effectiveness of the immune system and leaves individuals susceptible to opportunistic infections and tumors. AIDS is a pandemic (Kallings, J. Intern Med. 263, 218-243, 2008). In 2007, UNAIDS estimated: 33.2 million people worldwide had AIDS that year; AIDS killed an estimated 2.1 million people in the course of that year, including 330,000 children; 76% of those deaths occurred in sub-Saharan Africa. According to UNAIDS 2009 report, worldwide some 60 million people have been infected, with some 25 million deaths, and 14 million orphaned children in southern Africa alone since the epidemic began. There is no vaccine and few effective long-term therapeutics directed against HIV-1 infection and the subsequent development of AIDS.

Rev (13 kDa) is an essential regulatory protein of the HIV-1 virus which functions by binding to, and preventing splicing of, the viral mRNA, thereby facilitating transition to the late phase of the replication cycle (for review see Groom et al., J Gen Virol 90, 1303-18, 2009). Despite its importance, and considerable efforts directed at its elucidation, the structure of Rev remains unknown, due largely to the protein's strong propensity to polymerize into long filaments (Watts et al., J Struct Biol 121, 41-52, 1998; Wingfield et al., Biochemistry 30, 7527-34, 1991).

### SUMMARY

The preparation, characterization, and crystallization of a complex formed between Rev and a chimeric rabbit/human Fab selected by means of phage display are described herein. The preparation of the corresponding high-affinity single chain antibody fragment (scFv) is also described.

Provided in one embodiment is an anti-Rev antibody or a fragment thereof which maintains binding activity to HIV-1 Rev, which includes a V_{H} region and a V_{L} region. In various examples of this embodiment, the V_{H} region has a framework and comprises three CDRs: a first CDR comprising the amino acid sequence GFWLNW (positions 31-36 of SEQ ID NO: 2); a second CDR comprising the amino acid sequence AIYRGSGSEWYASWAKG (positions 50-66 of SEQ ID NO: 2); and a third CDR comprising the amino acid sequence AADTTDNGYFTI (positions 95-106 of SEQ ID NO: 2); or the V_{H} region has a sequence at least 90% identical to SEQ ID NO: 2. In various examples of this embodiment, the V_{L} region has a framework and comprises three CDRs: a first CDR comprising the amino acid sequence QASQSISSWLS (positions 25-35 of SEQ ID NO: 4); a second CDR comprising the amino acid sequence DASNLAS (positions 51-57 of SEQ ID NO: 4); and a third CDR sequence comprising the amino acid sequence LGGYPAASYRTA (positions 90-101 of SEQ ID NO: 4); or the V_{L} region has a sequence at least 90% identical to SEQ ID NO: 4. Examples of such anti-Rev antibodies and fragments include Fab SJS-R1 and SJS-R1 scFv. Also provided are isolated antibodies and antibody fragments that bind the same epitope as does the SJS-R1 Fab or scFv antibody or fragment.

Other embodiments provide isolated polynucleotides that encode the V_{H} and/or V_{L} region of the any of the anti-Rev antibodies or fragments described herein, including but not limited to SEQ ID NO: 1, 3, 5 and 8. Vectors comprising such polynucleotides, for instance under control of a promoter, are also provided, as are cells containing such vectors.

Pharmaceutical compositions that comprise one or more of the described anti-Rev antibodies or fragments thereof or encoding nucleotides are also described. Such pharmaceutical compositions optionally include one or more additional therapeutic agents.

Yet another embodiment is a method of inhibiting or preventing or reversing multimerization/polymerization of Rev, which method involves contacting Rev protein with an antibody or antibody fragment described herein, thereby preventing or reducing polymerization of Rev.

Another provided method is preventing or inhibiting replication of a lentivirus in a cell, which method comprises contacting a cell infected with the lentivirus with the anti-Rev antibody or fragment thereof described herein, thereby preventing or inhibiting replication of the lentivirus in the cell.

Also provided is a method of reducing infectivity or replication of a lentivirus, comprising contacting the lentivirus with the anti-Rev antibody or fragment thereof described herein, thereby reducing infectivity or replication of the lentivirus.

Yet another provided method is a method of inhibiting Rev function in a cell infected with a lentivirus, comprising contacting the cell with the anti-Rev antibody or fragment thereof described herein, thereby inhibiting Rev function in the cell.

Another embodiment provides a method of treating a disease or symptom associated with Rev expression or activity in an animal. This method comprises administering to the animal with said disease or symptom a therapeutically effective amount of the anti-Rev antibody or fragment described herein, thereby treating the disease or symptom.

In another embodiment, there is provided an article of manufacture comprising the anti-Rev antibody or fragment thereof described herein, for the treatment of an HIV infection or AIDS. Also provided are kits that comprise at least one anti-Rev antibody or fragment thereof described herein for the treatment of an HIV infection or AIDS.

Use of the anti-Rev antibodies or fragments thereof described herein can also be used to detect HIV or HIV infection, to diagnosis HIV infection, AIDS or ARC, or to monitor disease progression or recovery or treatment.

Another aspect of the invention relates to a method of inhibiting HIV infection of a cell, comprising contacting the cell with an effective amount of an antibody or functional portion/fragment thereof which binds to Rev.

Also encompassed by the present invention is a method of inhibiting (e.g., treating) HIV in a patient, comprising administering to the patient an effective amount of an antibody or functional portion thereof which binds to Rev.

Another aspect of the invention also relates to a method of preventing or inhibiting HIV infection in an individual, comprising administering to the individual an effective amount of an antibody or functional portion thereof which binds to Rev. According to the method, preventing HIV infection includes treatment in order to prevent (reduce or eliminate) infection of new cells in an infected individual or in order to prevent infection in an individual who may be, may have been or has been exposed to HIV. For example, individuals such as an HIV infected individual, a fetus of an HIV infected female, or a health care worker can be, treated according to the method of the present invention.

The foregoing and other objects, features, and advantages of the invention will become more apparent from the following detailed description, which proceeds with reference to the accompanying figures.

### BRIEF DESCRIPTION OF THE DRAWINGS

FIG. 1 shows the amino acid sequences of the variable domains of Fab SJS-R1. Shown are framework regions and complementary determining regions (CDR) of V_{κ} (positions 2-111 of SEQ ID NO: 4) and V_{H} (positions 1-117 of SEQ ID NO: 2).

FIG. 2 illustrates the purification of the SJS-R1 Fab by gel filtration chromatography. Fab from two cycles of Ni-Sepharose chromatography was applied to a Superdex S200 column (2.6 cm diameter×60 cm length) equilibrated with PBS containing 1 M urea. The main protein peak eluted at ˜250 mL. The smaller (conductivity) peak eluting at ˜290 mL is the salt peak (included volume). Inset (a) shows a Coomassie stained SDS-PAGE of non-reduced (Oxd) fractions taken across the main peak. The bar at the top corresponds to peak fractions also indicated with a bar in the chromatogram. The main band at ˜48 kDa corresponds to oxidized Fab. Inset (b) shows a Coomassie stained SDS-PAGE of purified Fab either not reduced (Oxd) or reduced (Red) with DTT. In the reduced sample, at the high protein loading employed, the light chain and the heavy chain fragment are not resolved and appear as a single band at ˜24 kDa.

FIG. 3 is a series of gels illustrating SDS-PAGE of purified Fab, scFv, and immune complexes with Rev. Proteins were either not reduced (panel a) or reduced (panels b and c), and then analyzed on 4-12% acrylamide gels and stained with Coomassie dye. Standards (Std) are labeled in units of kDa. Though the Fab in panel (a) is under loaded, it can be seen to be pure by examining the Fab/Rev lanes in panels (a) and (b). The ˜30 kDa band marked with an asterisk in panel (a) was identified as an oxidized dimer of Rev.

FIG. 4 is a series of charts used in calculating molecular weights of the Fab/Rev and scFv/Rev complexes. The molecular weight determinations by sedimentation equilibrium ultracentrifugation analysis are indicated in panels (a) to (c). The protein concentration profiles, represented by UV absorbance gradients at 280 nm, versus the radial distance, are indicated. The solid lines indicate data fitted using a single species model, and open circles represent the experimental data. The upper panels of each graph show the residuals (difference between the fitted and experimental values) as a function of radial position. Panel (d) shows the hydrodynamic radius (R) distribution for the Fab/Rev complex as determined by dynamic light scattering. Monodispersity is indicated by the main species which constitutes 97.9% of the protein. The molecular mass is estimated from the intensity of the scattered light.

FIG. 5 illustrates the surface plasmon resonance analysis of Fab binding to immobilized Rev. Duplicate, and in some cases triplicate, injections were made over the concentration range 8-23 nM Fab (right hand ordinate). The binding curves show the very low off-rates. The kinetic parameters kₐ, and k_{d} and the dissociation constant determined from these data (K_{d}=k_{d}/kₐ˜40 μM) are given.

FIG. 6 is two micrographs illustrating that the SJS-R1 Fab depolymerizes Rev filaments. Panel (a) shows an electron micrograph of negatively stained Rev filaments. The filaments have a stain penetrable lumen and are quite long, often extending over entire grid squares. Panel (b) shows that when the Fab and Rev are mixed in equimolar ratio the high affinity Fab binding rapidly depolymerizes the Rev filaments and results in the formation of small, uniformly sized complexes. Bar=100 nm; 50 nm in the inset.

FIG. 7 is a graph illustrating sedimentation velocity centrifugation analysis of the Fab/Rev complex. The Fab/Rev immune complex was centrifuged at 45,000 rpm, 20° C., with data collection every 10 minutes up to 3 hours. The insert shows protein absorbance at 280 nm as a function of radial distance. The movement of the single boundary toward the bottom of the cell is indicated by the arrow. The main panel shows the sedimentation coefficient distribution derived from data in the insert where the abscissa s* is the apparent sedimentation coefficient and has units of Svedbergs (S) (1 Svedberg=1×10⁻¹³ sec). The ordinate g(s*), is the distribution function of apparent sedimentation values, and has units of absorbance units per Svedberg (AU S⁻¹). The solid line is the data fitted to a single species and the points are the actual data. The distribution function, g(s*), is used to derive the concentration, sedimentation coefficient, and diffusion coefficient (Philo et al., Anal Biochem 279, 151-63, 2000). In the distribution plot the area under each peak gives the total amount of that species.

FIG. 8 is a photograph illustrating crystallization of the Fab/Rev complex. Several Fab/Rev crystals are shown, with an enlargement on the right. Crystals usually grew as long rods.

FIG. 9 is the X-ray diffraction pattern for a crystal of the Fab/Rev complex. Shown is a portion of a 1.0° oscillation diffraction pattern collected at the IO2 beamline at the Diamond Light Source (Didcot, UK). Concentric rings depict the 40.0, 20.0, 8.0, 4.0 and 3.2 Å resolution shells. In the final dataset data are measurable to 3.2 Å, with 6-fold non-crystallographic symmetry redundancy.

FIG. 10 is a three-dimensional illustration of the paratope of Fab SJS-R1 bound to a Rev monomer. Parts of the six CDRs (H1, H2, H3, L1, L2, and L3) are shown engaging an isosurface rendering of the Rev monomer.

### SEQUENCES

The nucleotide and amino acid sequences listed herein and/or in the accompanying Sequence Listing are shown using standard letter abbreviations for nucleotide bases, and three letter code (as defined in 37 C.F.R. 1.822) or one letter code for amino acids. Only one strand of each nucleic acid sequence is shown, but the complementary strand is understood as included by any reference to the displayed strand. In the present application:

SEQ ID NO: 1 shows the nucleic acid sequence encoding Rev Fab SJS-R1 heavy chain. This encodes sequence that overlaps positions 2-117 of SEQ ID NO: 2.

SEQ ID NO: 2: shows the amino acid sequence of Rev Fab SJS-R1 heavy chain (V_{H}) (CDRH1 is positions 31-36; CDRH2 is positions 50-66, and CDRH3 is positions 95-106).

SEQ ID NO: 3 shows the nucleic acid sequence encoding Rev Fab SJS-R1 light chain. This encodes sequence that overlaps positions 1-110 of SEQ ID NO: 4.

SEQ ID NO: 4: shows the amino acid sequence of Rev Fab SJS-R1 light chain (CDRL1 is positions 25-35; CDRL2 is positions 51-57, and CDRL3 is positions 90-101).

SEQ ID NO: 5 is the nucleic acid sequence shared by the six positive Fab clones (Example 1); this gene has been labeled SJS-R1. The two initiating ATG codons are underlined (at positions 1-3 and 751-753); the sequences that encode the mature SJS-R1 (V_{L} at positions 67-718 followed by the stop codon taa and V_{H} at positions 817-1509, including a polyHis tag, followed by the stop codon tga) are shown in upper case. The italic sequence is non-translated, separating the heavy and light chains; the remaining lower case sequence encodes the ompA secretory peptide used to direct secretion of the Fab proteins in the E. coli synthesis system; the secretory peptides are removed during production of the proteins:

atgaaaaagacagctatcgcgattgcagtggcactggctggtttcgct
accgtggcccaggcggccGAGCTCGTGATGACCCAGACTCCATCCTCC
GTGTCTGAACCTGTGGGAGGCACAGTCACCATCAAGTGCCAGGCCAGT
CAGAGCATTAGCAGTTGGTTATCCTGGTATCAGCAGAAACCAGGGCAG
CCTCCCAAGCTCCTGATCTACGATGCATCCAATCTGGCATCTGGGGTC
CCGTCGCGATTTTATGGGCAGTGGGTCTGGGACAGAGTACACTCTCAC
CATCAGCGGCGTGCAGCGTGAGGATGCTGCCACCTACTACTGTCTAGG
TGGTTATCCTGCTGCTTCTTATCGAACTGCTTTCGGCGGAGGGACCGA
GCTGGAGATCATACGAACTGTGGCTGCACCATCTGTCTTCATCTTCCC
GCCATCTGATGAGCAGTTGAAATCTGGAACTGCCTCTGTTGTGTGCCT
GCTGAATAACTTCTATCCCAGAGAGGCCAAAGTACAGTGGAAGGTGGA
TAACGCCCTCCAATCGGGTAACTCCCAGGAGAGTGTCACAGAGCAGGA
CAGCAAGGACAGCACCTACAGCCTCAGCAGCACCCTGACGCTGAGCAA
AGCAGACTACGAGAAACACAAAGTCTACGCCTGCGAAGTCACCCATCA
GGGCCTGAGTTCGCCCGTCACAAAGAGCTTCAACAGGGGAGAGTGTta
attctagataattaattaggaggaatttaaaatgaaatacctattgcc
tacggcagccgctggattguattactcgctgcccaaccagccatggcc
CAGGAGCAGCTGGTGGAGTCCGGGGGTCGCCTGGTCACGCCTGGGACAG
CCCTGACACTCACCTGCAAAGTCTCTGGATTCTCCCTCAGTGGCTTCT
GGCTGAACTGGGTCCGCCAGGCTCCAGGGAAGGGGCTGGAGTGGGTC
GGAGCCATTTACAGAGGTAGTGGTAGTGAATGGTACGCGAGCTGGGC
AAAAGGCCGATTCACTATCTCCGATACCTCGACCACGGTGACTCTGA
AACTGACCAGTCCAACAACCGAGGACACGGCCACCTATTTCTGTGCC
GCTGATACTACTGATAATGGGTACTTTACCATCTGGGGCCCAGGCACC
CTGGTCACCGTCTCCTCAGCCTCCACCAAGGGCCCATCGGTCTTCCCC
TTGGCCCCGTCGGCCAAGAGCACCTCTGGGGGCACAGCGGCCCTGGG
CTGCCTGGTCAAGGACTACTTCCCCGAACCGGTGACGGTGTCGTGGA
ACTCAGGCGCCCTGACCAGCGGCGTGCACACCTTCCCGGCTGTCCTAC
AGTCCTCAGGACTCTACTCCCTCAGCAGCGTGGTGACCGTGCCCTCCA
GCAGCTTGGGCACCCAGACCTACATCTGCAACGTGAATCACAAGCCC
AGCAACACCAAGGTGGACAAGAAAGCAGAGCCCAAATCTTGTGACA
AAACTAGGGGTCATCATCACCATCACCATtga

SEQ ID NO: 6 is the amino acid sequence of the SJS-R1 REV Fab light chain precursor, encoded by positions 1-720 of SEQ ID NO: 5 (including the stop codon).

SEQ ID NO: 7 is the amino acid sequence of the SJS-R1 REV Fab heavy chain+polyHis, encoded by positions 817-1512 of SEQ ID NO: 5 (including the stop codon).

SEQ ID NO: 8 shows the nucleic acid sequence encoding SJS-R1 scFv precursor:

SEQ ID NO: 9 shows the amino acid sequence of the SJS-R1 scFv precursor. The components of the scFv precursor are as follows: ompA (positions 1 to 21)-Vκ (positions 22 to 132)-linker (positions 133 to 150)-V_{H} (positions 151 to 267)-polyHis (positions 268 to 273).

SEQ ID NO: 10 shows the amino acid sequence of the mature SJS-R1 scFv. The components of the mature scFv are as follows: Vic (positions 1 to 111)-linker (positions 112 to 130)-V_{H} (positions 131 to 246)-polyHis (positions 247 to 252).

SEQ ID NO: 11 is a sequence encoding a cell penetrating peptide.

### DETAILED DESCRIPTION

### I. Abbreviations

AIDS acquired immune deficiency syndrome
CDR complementarity-determining region
CPP cell penetrating peptide
FR framework region
HAART highly active anti-retroviral therapy
HIV human immunodeficiency virus
NNRTI non-nucleoside reverse transcriptase inhibitors
pM picomolar
Rev regulator of virion

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below.

All publications, patent applications, patents, and other references mentioned herein are incorporated by reference in their entirety. All GenBank Accession numbers mentioned herein are incorporated by reference in their entirety as present in GenBank on Feb. 3, 2011. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Antibody: Immunoglobulin molecules and immunologically active portions of immunoglobulin molecules, e.g., molecules that contain an antigen binding site that specifically binds (immunoreacts with) an antigen. It is known that the antigen-binding function of an antibody can be performed by fragments of a naturally occurring antibody. Such antigen-binding fragments are also intended to be designated by the term “antibody” herein, unless the context makes it clear that another meaning is intended.

Antibody fragment (fragment with specific antigen binding): Various fragments of antibodies have been defined, including Fab, (Fab′)₂, Fv, and single-chain Fv (scFv). These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain or equivalently by genetic engineering; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction or equivalently by genetic engineering; (4) F(Ab′)₂, a dimer of two Fab′ fragments held together by disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Additional antibody based molecules include intrabodies (intracellular antibodies), transbodies (delivered as protein from outside of a cell—that is, systemically) nanobodies, minibodies, diabodies, and so forth. Methods of making these fragments and other antibody-based molecules are routine in the art.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 8 amino acids (such as about 8-50 or 8-23 amino acids) in a unique spatial conformation. Methods of determining spatial conformation of epitopes include, for example, X-ray crystallography and two-dimensional nuclear magnetic resonance.

Antigenic surface: A surface of a molecule, for example a protein such as a Rev protein or polypeptide, capable of eliciting an immune response. An antigenic surface includes the defining features of that surface, for example the three-dimensional shape and the surface charge. An antigenic surface includes both surfaces that occur on Rev polypeptides as well as surfaces of compounds that mimic the surface of a Rev polypeptide (mimetics). In some examples, an antigenic surface include all or part of the surface of Rev that binds to (or is bound by) SJS-R1 Fab or scFv.

Atomic Coordinates or Structure coordinates: Mathematical coordinates derived from mathematical equations related to the patterns obtained on diffraction of a monochromatic beam of X-rays by the atoms (scattering centers) such as an antigen, or an antigen in complex with an antibody. In some examples that antigen can be Rev, a Rev:antibody complex, or combinations thereof in a crystal. The diffraction data are used to calculate an electron density map of the repeating unit of the crystal. The electron density maps are used to establish the positions of the individual atoms within the unit cell of the crystal. In one example, the term “structure coordinates” refers to Cartesian coordinates derived from mathematical equations related to the patterns obtained on diffraction of a monochromatic beam of X-rays, such as by the atoms of Rev (or Rev complexed with SJS-R1 Fab) in crystal form.

Those of ordinary skill in the art understand that a set of structure coordinates determined by X-ray crystallography is not without standard error. For the purpose of this disclosure, any set of structure coordinates that have a root mean square deviation of protein backbone atoms (N, Cα, C and O) of less than about 1.0 Angstroms when superimposed, such as about 0.75, or about 0.5, or about 0.25 Angstroms, using backbone atoms, shall (in the absence of an explicit statement to the contrary) be considered identical.

Binding or interaction: A direct or indirect association between two substances or molecules, such as the hybridization of one nucleic acid molecule to another (or itself), or a specific association between two or more proteins, such as an antibody and its cognate antigen or components of a multi-protein complex.

Binding affinity: Affinity of an antibody or antigen binding fragment thereof for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity is at least about 1×10⁻⁸ M. In other embodiments, a high binding affinity is at least about 1.5×10⁻⁸, at least about 2.0×10⁻⁸, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M.

cDNA: A DNA molecule lacking internal, non-coding segments (e.g., introns) and regulatory sequences that determine transcription. By way of example, cDNA may be synthesized in the laboratory by reverse transcription from messenger RNA extracted from cells.

Chimeric antibody: An antibody which includes sequences derived from two different antibodies, which typically are of different species. In some examples, a chimeric antibody includes one or more CDRs and/or framework regions from one human antibody and CDRs and/or framework regions from another human antibody.

Complementarity-determining region (CDR): The CDRs are three hypervariable regions within each of the variable light (V_{L}) and variable heavy (V_{H}) regions'of an antibody molecule that form the antigen-binding surface that is complementary to the three-dimensional structure of the bound antigen. Proceeding from the N-terminus of a heavy or light chain, these complementarity-determining regions are denoted as “CDR1,” “CDR2,” and “CDR3,” respectively; these abbreviations can also include a designation of light or heavy chain, thus “CDRH1” or “CDRL1”. CDRs are involved in antigen-antibody binding.

An antigen-binding site, therefore, may include six CDRs, comprising the CDR regions from each of a heavy and a light chain variable region. Alteration of a single amino acid within a CDR region can destroy the affinity of an antibody for a specific antigen (see Abbas et al., Cellular and Molecular Immunology, 4th ed. 143-5, 2000), though there are recognized methods (including methods described or referenced herein) that can be used to test whether any particular amino acid change impacts the specificity of an antibody. The locations of the CDRs have been precisely defined, e.g., by Kabat et al., Sequences of Proteins of Immunologic Interest, U.S. Department of Health and Human Services, 1983.

Conservative variation: A phrase that denotes the replacement of an amino acid residue by another, biologically similar residue; a.k.a. conservative amino acid substitution. Examples of conservative variations include the substitution of one hydrophobic residue (such as isoleucine, valine, leucine or methionine) for another, or the substitution of one polar residue for another (such as the substitution of arginine for lysine, glutamic for aspartic acid, or glutamine for asparagine, and the like). The term “conservative variation” also includes the use of a substituted amino acid in place of an unsubstituted parent amino acid. Non-limiting examples of conservative amino acid substitutions include: Ala for Ser; Arg for Lys; Asn for Gln or His; Asp for Glu; Cys for Ser; Gln for Asn; Glu for Asp; His for Asn or Gln; Ile for Leu or Val; Leu for Ile or Val; Lys for Arg, Gln or Glu; Met for Leu or Ile; Phe for Met, Leu or Tyr; Ser for Thr; Thr for Ser; Trp for Tyr; Tyr for Trp or Phe; and Val for Ile or Leu.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A “control” refers to a sample or standard used for comparison with an experimental sample. In some embodiments, the control is a sample obtained from a healthy subject (such as a subject without viral infection or an associated syndrome or condition, for instance without AIDS). In some embodiments, the control is a historical control or standard reference value or range of values (such as a previously tested control sample, such as a group of subjects with a viral infection such as HIV, or group of samples from subjects that do not have AIDS or are not infected with HIV). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval).

Derivative: A compound or portion of a compound that is derived from or is theoretically derivable from a parent compound, for example if at least one atom is replaced with another atom or group of atoms. Derivatives also include compounds to which at least one atom or functional group is added or removed, rather than replacing an atom or functional group of the parent compound.

DNA (deoxyribonucleic acid): DNA is a long chain polymer that contains the genetic material of most living organisms (the genes of some viruses are made of ribonucleic acid (RNA)). The repeating units in DNA polymers are four different nucleotides, each of which includes one of the four bases (adenine, guanine, cytosine and thymine) bound to a deoxyribose sugar to which a phosphate group is attached. Triplets of nucleotides (referred to as codons) code for each amino acid in a polypeptide, or for a stop signal. The term “codon” is also used for the corresponding (and complementary) sequences of three nucleotides in the mRNA into which the DNA sequence is transcribed.

Epitope: Any antigenic determinant on an antigen to which the paratope of an antibody binds. Epitopic determinants usually consist of chemically active surface groupings of molecules such as amino acids or sugar side chains, and usually have specific three dimensional structural characteristics, as well as specific charge characteristics.

Framework region (FR): Relatively conserved sequences flanking the three highly divergent complementarity-determining regions (CDRs) within the variable regions of the heavy and light chains of an antibody. Hence, the variable region of an antibody heavy or light chain consists of a FR and three CDRs. Some FR residues may contact bound antigen; however, FRs are primarily responsible for folding the variable region into the antigen-binding site, particularly the FR residues directly adjacent to the CDRs. Without being bound by any single theory, the framework region of an antibody serves to position and align the CDRs. The sequences of the framework regions of different light or heavy chains are relatively conserved within a species. A “human” framework region is a framework region that is substantially identical (about 85% or more, usually 90-95% or more) to the framework region of a naturally occurring human immunoglobulin.

Fusion protein: A protein that has two (or more) parts fused (joined, usually by way of a covalent bond) together, which are not found joined together in nature. In general, the two domains are genetically fused together, in that nucleic acid molecules that encode each protein part or domain are functionally linked together, for instance directly or by a linker oligonucleotide, thereby producing a single fusion-encoding nucleic acid molecule. The translated product of such a fusion-encoding nucleic acid molecule is the fusion protein. Fusion proteins are sometimes referred to as “chimeric” proteins, because they have fused parts derived from different origins.

HIV-1: The human immunodeficiency virus type-1; HIV-1 includes but is not limited to extracellular virus particles and the forms of HIV-1 found in HIV-1 infected cells.

HIV-1 infection: The introduction of HIV-1 genetic information into a target cell, such as by fusion of the target cell membrane with HIV-1 or an HIV-1 envelope glycoprotein and cell. The target cell may be a cell of (or in) a subject, such as for instance a human subject.

Hybridization: Nucleic acid molecules that are complementary to each other hybridize by hydrogen bonding, which includes Watson-Crick, Hoogsteen or reversed Hoogsteen hydrogen bonding between complementary nucleotide units. For example, adenine and thymine are complementary nucleobases that pair through formation of hydrogen bonds. “Complementary” refers to sequence complementarity between two nucleotide units. For example, if a nucleotide unit at a certain position of an oligonucleotide is capable of hydrogen bonding with a nucleotide unit at the same position of a DNA or RNA molecule, then the oligonucleotides are complementary to each other at that position. The oligonucleotide and the DNA or RNA are complementary to each other when a sufficient number of corresponding positions in each molecule are occupied by nucleotide units which can hydrogen bond with each other.

“Specifically hybridizable” and “complementary” are terms that indicate a sufficient degree of complementarity such that stable and specific binding occurs between the oligonucleotide and the DNA or RNA or PNA target. An oligonucleotide need not be 100% complementary to its target nucleic acid sequence to be specifically hybridizable. An oligonucleotide is specifically hybridizable when binding of the oligonucleotide to the target DNA or RNA molecule interferes with the normal function of the target DNA or RNA, and there is a sufficient degree of complementarity to avoid non-specific binding of the oligonucleotide to non-target sequences under conditions in which specific binding is desired, for example under physiological conditions in the case of in vivo assays, or under conditions in which the assays are performed.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. in Molecular Cloning: A Laboratory Manual, Cold Spring Harbor Laboratory Press (1989), chapters 9 and 11, herein incorporated by reference.

Immunoglobulin: A protein including one or more polypeptides substantially encoded by immunoglobulin genes. The recognized immunoglobulin genes include the two light chains kappa (κ) and lambda (λ), and the heavy chains alpha (α; IgA), gamma (γ; IgG₁, IgG₂, IgG₃, IgG₄), delta (δ; IgD), epsilon (ε; IgE) and mu (μ; IgM) constant region genes, as well as the myriad immunoglobulin variable region genes. Full-length immunoglobulin light chains are generally about 25 kD or 214 amino acids in length. Full-length immunoglobulin heavy chains are generally about 50 kD or 446 amino acid in length. Light chains are encoded by a variable region gene at the amino terminus (about 110 amino acids in length) and a kappa or lambda constant region gene at the carboxy terminus. Heavy chains are similarly encoded by a variable region gene (about 116 amino acids in length) and one of the other constant region genes. Inside both the light and the heavy chain, the variable and constant regions are joined by a “J” region of about 12 amino acids or more, whilst only the heavy chains include a “D” region of about 10 amino acids.

The basic structural unit of an antibody is generally a tetramer that consists of two identical pairs of immunoglobulin chains, each pair having one light and one heavy chain.

In each pair, the light and heavy chain variable regions bind to an antigen, and the constant regions mediate effector functions. Immunoglobulins also exist (and can be engineered to exist) in a variety of other forms including, for example, Fv, Fab, and (Fab′)₂, as well as bifunctional hybrid antibodies and single chains (e.g., Lanzavecchia et al., Eur. J. Immunol. 17:105, 1987; Huston et al., Proc. Natl. Acad. Sci. U.S.A., 85:5879-5883, 1988; Bird et al., Science 242:423-426, 1988; Hood et al., Immunology, Benjamin, N.Y., 2nd ed., 1984; Hunkapiller and Hood, Nature 323:15-16, 1986). Immunoglobulins and certain variants thereof are known, and many have been prepared in recombinant cell culture (e.g., see U.S. Pat. Nos. 4,745,055 and 4,444,487; WO 88/03565; EP 256,654; EP 120,694; EP 125,023; Falkner et al., Nature 298:286, 1982; Morrison, J. Immunol. 123:793, 1979; Morrison et al., Ann. Rev. Immunol. 2:239, 1984).

An immunoglobulin light or heavy chain variable region includes a framework region interrupted by three hypervariable regions, also called CDRs (see, Sequences of Proteins of Immunological Interest, Kabat et al., U.S. Department of Health and Human Services, 1983). From the amino-terminal region and to the carboxy-terminal region, the variable domains both of the light chain and of the heavy chain comprise and alternation of FR and CDR regions: FR, CDR, FR, CDR, FR, CDR, FR. Both the heavy chain and the light chain are characterized by three CDRs, respectively CDR1 (or more specifically, CDRH1 or CDRL1), CDR2 (CDRH2 or CDRL2), CDR3 (CDRH3 or CDRL3). As noted herein, the CDRs are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse or rabbit monoclonal antibody can be joined to human constant segments, such as kappa and gamma 1 or gamma 3. In one example, a therapeutic chimeric antibody is thus a hybrid protein composed of the variable or antigen-binding domain from a mouse or rabbit antibody and the constant or effector domain from a human antibody, although other mammalian species can be used. Alternatively, the variable region can be produced by molecular techniques. Methods of making chimeric antibodies are well known in the art, e.g., see U.S. Pat. No. 5,807,715, which is herein incorporated by reference.

A “humanized” immunoglobulin is an immunoglobulin including a human framework region and one or more CDRs from a non-human (such as a mouse, rat, or synthetic) immunoglobulin. The non-human immunoglobulin providing the CDR(s) is termed a “donor” and the human immunoglobulin providing the framework is termed an “acceptor.” In one embodiment, all the CDRs in a humanized immunoglobulin are from the donor immunoglobulin. Constant regions need not be present, but if they are, they will be substantially similar to human immunoglobulin constant regions, e.g., at least about 85-90%, such as about 95% or more identical. Hence, all parts of a humanized immunoglobulin, except possibly the CDRs, are substantially similar (or identical) to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions include those described herein. Humanized immunoglobulins can be constructed by means of genetic engineering, e.g., see U.S. Pat. No. 5,225,539 and U.S. Pat. No. 5,585,089, which are incorporated by reference herein.

A human antibody is an antibody wherein the light and heavy chain genes are of human origin. Human antibodies can be generated using methods known in the art. For instance, human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., WO91/17271; WO92/001047; and WO92/20791, which are herein incorporated by reference), or selected from a human combinatorial monoclonal antibody library (e.g., from MorphoSys AG, Martinsried/Planegg, Germany). Human antibodies can also be prepared by using transgenic animals carrying a human immunoglobulin gene (e.g., see WO93/12227; and WO91/10741, which are herein incorporated by reference).

Immune response: A response of a cell of the immune system, such as a B cell or T cell to a stimulus. In one embodiment, the response is specific for a particular antigen (an “antigen-specific response”).

Inhibiting HIV infection: Reduction of the amount of HIV genetic information introduced into a target cell or target cell population in the presence of a composition (e.g., a therapeutic composition) or other treatment, compared to the amount that would be introduced into a similar cell or cell population in the absence of the composition or treatment.

Intrabody: An antibody that is expressed within a cell and directed against an intracellular target molecule. Optionally, the intrabody is expressed within or targeted to a specific subcellular compartment as directed by a localization signal genetically fused to N- or C-terminus of encoded antibody or antibody fragment (e.g., scFv or other binding fragment). For a review of uses of intrabodies in therapy against infectious diseases, such as HIV infection, see Rondon & Marasco (Annu. Rev. Microbiol. 51:257-83, 1997). Design and expression of a representative intrabody based on a scFv directed against HIV gp120 has been described, for instance, in Marasco et al. (Proc. Nat'l Acad. Sci U.S.A. 90:7889-7893, 1993).

Isolated: An isolated biological component (such as a nucleic acid, peptide, protein, or a compound, such as a small organic or inorganic molecule) has been substantially separated, produced apart from, or purified away from other biological components in the cell of the organism in which the component naturally occurs, for example the separation of a peptide from a sample, such as saliva, urine, serum or blood. The term also embraces nucleic acids, proteins and compounds prepared by recombinant expression in a host cell, as well as chemically synthesized peptides, nucleic acids, and other compounds. It is understood that the term “isolated” does not require that the component is free of trace contamination, and thus the term includes molecules that are at least 50% isolated, such as at least 50%, 55%, 60%, 65%, 70%, 75%, 80%, 85%, 90%, 95%, 98%, 99%, or even 100% isolated.

In vitro amplification: Techniques that increase the number of copies of a nucleic acid molecule in a sample or specimen. An example of in vitro amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid.

The product of in vitro amplification may be characterized by electrophoresis, restriction endonuclease cleavage patterns, oligonucleotide hybridization or ligation, and/or nucleic acid sequencing, using standard techniques.

Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Isolated: An “isolated” component (for instance, a biological component, such as a nucleic acid molecule, peptide, protein or organelle) has been substantially separated or purified away from other components in which the component naturally occurs. In the case of a biological component, the isolated component has been substantially separated or purified away from other components in the reaction vessel used to make the component, or the cells of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids or peptides.

Label: A composition detectable by spectroscopic, photochemical, biochemical, immunochemical, chemical or other means. Typical labels include fluorescent proteins or protein tags, fluorophores, radioactive isotopes (including for instance ³²P), ligands, biotin, dioxigenin, chemiluminescent agents, electron-dense reagents (such as metal sols and colloids), and enzymes (e.g., for use in an ELISA), haptens, and proteins or peptides (such as epitope tags) for which antisera or monoclonal antibodies are available. Methods for labeling and guidance in the choice of labels useful for various purposes are discussed, e.g., in Sambrook et al., in Molecular Cloning: A Laboratory Manual, Cold Spring Harbor Laboratory Press (1989) and Ausubel et al., in Current Protocols in Molecular Biology, Greene Publishing Associates and Wiley-Intersciences (1987). A label often generates a measurable signal, such as radioactivity, fluorescent light or enzyme activity, which can be used to detect and/or quantitate the amount of labeled molecule.

Linker: A linker is a “chemical arm” between two moieties or domains in a molecule. Linkers may be used to join otherwise separate molecule moieties through a chemical reaction. The term “linker” also refers to the part of a fusion molecule between two moieties or subsections. In some embodiments, the linker in a fusion molecule, such as a fusion protein, is added by recombinant DNA techniques; in other embodiments, it is added through chemical means, such as cross-linking reactions or other in vitro chemical synthesis.

Many sorts of different chemical structures may constitute a linker (e.g., a peptide-to-peptide bond, a covalent bond between two protein domains, such as an amide, ester, or alkylamino linkages, or a single translated protein having two moieties “linked” by a series of residues). One non-limiting example of a linker is a synthetic sequence of amino acids. Other examples of linkers include streptavidin linkage, a straight or branched chain aliphatic group, particularly an alkyl group, such as C₁-C₂₀, optionally containing within the chain double bonds, triple bonds, aryl groups or heteroatoms such as N, O or S. Substituents on a diradical moiety can include C₁-C₆ alkyl, aryl, ester, ether, amine, amide, or chloro groups.

Additional types of bond combinations that may serve to link molecules are amino with carboxyl to form amide linkages, carboxy with hydroxy to form ester linkages or amino with alkyl halides to form alkylamino linkages, thiols with thiols to form disulfides, thiols with maleimides, and alkylhalides to form thioethers, for instance. Hydroxyl, carboxyl, amino and other functionalities, where not present may be introduced by known methods. Examples of specific linkers can be found, for instance, in Hennecke et al. (Protein Eng. 11: 405-410, 1998); and U.S. Pat. Nos. 5,767,260 and 5,856,456.

Linkers may vary in length in different embodiments, depending for instance on the molecular moieties being joined, on their method of synthesis, and on the intended function(s) of the fusion molecule. Linkers may be repetitive or non-repetitive.

Linkers in some embodiments consist of an amino acid sequence that covalently links two polypeptide domains to each other. One classical repetitive peptide linker used in the production of single chain Fvs (scFvs) is the (Gly₄Ser)₃ (or (G₄S)₃ or (G₄S)₃) linker. Non-repetitive linkers have been produced, and methods for the random generation of such linkers are known (Hennecke et al., Protein Eng. 11:405-410, 1998). In addition, linkers may be chosen to have more or less secondary character (e.g. helical character, U.S. Pat. No. 5,637,481) depending on the conformation desired in the final fusion molecule. The more secondary character a linker possesses, the more constrained the structure of the final fusion molecule will be. Therefore, substantially flexible linkers that are substantially lacking in secondary structure allow flexion of the fusion molecule at the linker.

Moiety: A part or portion of a molecule having a characteristic chemical, biochemical, structural and/or pharmacological property or function. As used herein, the term moiety refers to a subpart of a molecule (for instance, a protein) that retains an independent biochemical or structural activity from the remainder of the molecule, for instance the ability to generate a detectable signal such as fluorescence, or to bind or associate or interact with a target. A single molecule may have multiple moieties, each having an independent function.

Monoclonal antibody (mAb): An antibody produced by a single clone of B-lymphocytes or by a cell into which a single light and a single heavy chain have been transfected. Optionally, light and heavy chain of a monoclonal antibody may originate from different B-lymphocytes. Monoclonal antibodies are produced by methods known to those of skill in the art, for instance by making hybrid antibody-forming cells from a fusion of myeloma cells with immune spleen cells.

Nucleotide: A term that includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: A linear single-stranded polynucleotide sequence ranging in length from 2 to about 5,000 bases, for example a polynucleotide (such as DNA or RNA) which is at least 6 nucleotides, for example at least 10, 12, 15, 18, 20, 25, 50, 100, 200, 1,000, or even 5,000 nucleotides long. Oligonucleotides are often synthetic but can also be produced from naturally occurring polynucleotides.

An oligonucleotide analog refers to molecules that function similarly to oligonucleotides but have non-naturally occurring portions or components. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules. Such analog molecules may also bind to or interact with polypeptides or proteins.

Operatively (or operably) linked: A juxtaposition wherein the components so described are in a relationship permitting them to function in their intended manner. For instance, a first nucleic acid sequence is operably linked with a second nucleic acid sequence when the first nucleic acid sequence is placed in a functional relationship with the second nucleic acid sequence. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (i.e., ATG) in front of (upstream or 5′ to) a protein-encoding sequence, a splicing signal for introns, maintenance of the correct reading frame of that gene to permit proper translation of mRNA, and secretion signals, and stop codons. The term “control sequences” is intended to included, at a minimum, components whose presence can influence expression, and can also include additional components whose presence is advantageous, for example, leader sequences and fusion partner sequences. Expression control sequences can include a promoter.

Open reading frame (ORF): A series of nucleotide triplets (codons) coding for amino acids without any internal termination codons. These sequences are usually translatable into a peptide.

Paratope: The part of an antibody which recognizes the epitope of an antigen; the antigen-binding site of an antibody. It is a small portion (of about 15-22 amino acids) of the Fv domain antibody's. The three-dimensional structure of a paratope includes portions of both heavy and light chains.

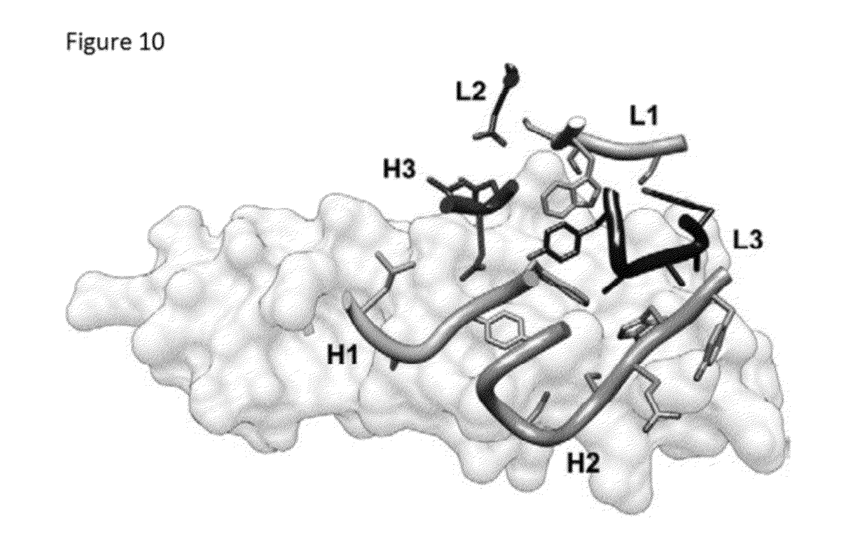

FIG. 10 shows the paratope of the SJS-R1 bound to Rev.

Parenteral: Administered outside of the intestine, for example, not via the alimentary tract. Generally, parenteral formulations are those that will be administered through any possible mode except ingestion. This term especially refers to injections, whether administered intravenously, intrathecally, intramuscularly, intraperitoneally, intraarticularly, or subcutaneously, and various surface applications including intranasal, intradermal, and topical application.

Pharmaceutically acceptable vehicles/carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent or drug: A chemical compound or composition (e.g., a composition comprising an antibody or antigen-binding fragment thereof) capable of inducing a desired therapeutic or prophylactic effect when properly administered to a subject or a cell, for example when it is incubated or contacted with a cell. “Incubating” includes exposing for a sufficient amount of time for a drug to interact with a cell or cellular component, such as a protein. “Contacting” includes incubating a compound such as a drug, in solid or in liquid form, with a cell. An example of a desired effect is an anti-viral effect, which inhibits a virus from replicating or infecting cells. Similarly, an “anti-retroviral agent” is an agent that specifically inhibits a retrovirus from replicating or infecting cells.

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “residue” or “amino acid residue” includes reference to an amino acid that is incorporated into a protein, polypeptide, or peptide.

Conservative amino acid substitutions (a.k.a., conservative variations) are those substitutions that, when made, least interfere with the properties of the original protein, that is, the structure and especially the function of the protein is conserved and not significantly changed by such substitutions. Non-limiting examples of conservative amino acid substitutions include: Ala for Ser; Arg for Lys; Asn for Gln or His; Asp for Glu; Cys for Ser; Gln for Asn; Glu for Asp; His for Asn or Gln; Ile for Leu or Val; Leu for Ile or Val; Lys for Arg, Gln or Glu; Met for Leu or Ile; Phe for Met, Leu or Tyr; Ser for Thr; Thr for Ser; Trp for Tyr; Tyr for Trp or Phe; and Val for Ile or Leu.

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Preventing, treating or ameliorating a disease: “Preventing” a disease (such as metastatic melanoma) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease; treating and ameliorating are not mutually exclusive terms.

Probes and primers: A probe comprises an isolated nucleic acid capable of hybridizing to a target nucleic acid. A detectable label or reporter molecule can be attached to a probe or primer. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998). In some embodiments, an “oligonucleotide” is a probe or primer.

In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally at least 15 nucleotides in length, such as at least 15, at least 16, at least 17, at least 18, at least 19, least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, or more contiguous nucleotides complementary to the target nucleic acid molecule, such as 15-70 nucleotides, 15-60 nucleotides, 15-50 nucleotides, 15-40 nucleotides, or 15-30 nucleotides.

Primers are short nucleic acid molecules, for instance DNA oligonucleotides 15 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50, 55, 60, 65, 70 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, or more contiguous nucleotides complementary to the target nucleic acid molecule to be amplified, such as a primer of 15-70 nucleotides, 15-60 nucleotides, 15-50 nucleotides, 15-40 nucleotides or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and primers can be readily prepared based on the nucleic acid molecules provided herein. It is also appropriate to generate probes and primers based on fragments or portions of these disclosed nucleic acid molecules, for instance regions that encompass the identified mutations of interest. PCR primer pairs can be derived from a known sequence by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Purified: The term purified does not require absolute purity; rather, it is intended as a relative term. Thus, for example, a purified antibody or other protein/peptide preparation is one in which the antibody (or other specified protein or peptide) is more enriched than in its natural environment within a cell or a laboratory production vessel. Preferably, a preparation is purified such that the “purified” compound represents at least 50% of the total content of the preparation, for example, at least 50% by weight.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Retroviruses: Viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of retroviruses that cause slow (“lenti”) diseases. The lentiviruses include but are not limited to human immunodeficiency virus (HIV) type 1 and type 2 (HIV-1 and HIV-2), simian immunodeficiency virus (SIV), and feline immunodeficiency virus (Hy), which are immunodeficiency viruses.

HIV is a retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by an HIV virus, wherein the infection may be confirmed by antibody or western blot studies. Laboratory findings associated with this disease include a progressive decline in T-helper cells. The term “HIV disease” is a generic term that includes AIDS.

Rev: Rev (“regulator of virion”) is a HIV gene that encodes a trans-acting nuclear protein that allows fragments of HIV mRNA that contain a Rev Response Element (RRE) to be exported from the nucleus of an infected cell to the cytoplasm. In the absence of the rev gene or its expression, the host RNA splicing machinery in the nucleus splices HIV transcripts so that only the smaller, regulatory proteins are produced; in the presence of rev, HIV RNA is exported from the nucleus before it can be spliced, which permits production of HIV structural proteins and the RNA genome. This permits a positive feedback loop which provides time-dependent regulation of replication (Strebel, AIDS 17 Suppl 4:S25-34, 2003). One representative Rev sequence is GenBank Accession P04616, HIV-1 Rev from HV1B1 (isolate BH10). Additional representative Rev sequences from HIV-1 can be found, for instance, in GenBank Accession numbers sp|P04616.1|REV_HV1B1, sp|P69718.1|REV_HV1H3, sp|P04620.1|REV_HV1BR, pir∥VKLJH3, sp|P04325.1|REV_HV112, pdb|2X7L|M, dbj|BAA12993.1|, gb|ACR51454.1|, gb|AAK08487.1|AF324493_{—}6, gb|ABI79804.1|, dbj|BAA13001.1|, gb|AAT11242.1|, gb|ABI79918.1|, gb|ABI80040.1|, gb|AAT11215.1|, ref|NP_{—}057854.1↑, sp|Q70624.1|REV_HV1LW, gb|ABI79856.1|, gb|ABI79796.1|, gb|ACQ84396.1|, gb|ABI79730.1|, gb|ABI79935.1|, gb|AAC28450.1|, gb|ABI79892.1|, gb|ABI79822.1|, gb|ACR51477.1|, gb|AAF22336.1|AF193277_{—}9, gb|ABS20069.1|, gb|AAV28701.1|, gb|ABI79970.1|, gb|ABI79847.1|, gb|AAC54647.1|, gb|AAX39505.1|, gb|ABI79979.1|, gb|AAB23299.1|, gb|ABI79787.1|, gb|AAC33099.1|, gb|ABI79874.1|, gb|ABI79772.1|, gb|ABB29381.1|, dbj|BAH96519.1|, gb|ABI79839.1|, dbj|BAH96528.1|, gb|AAO63192.1|, gb|ABI79926.1|, gb|AAX39506.1|, gb|ABI79909.1|, gb|ADM64161.1|, gb|AAQ97466.1|, and gb|AAL78495.1|AF414006_{—}7, each of which is incorporated herein as Feb. 3, 2011.

As all lentiviruses encode a Rev protein and employ the Rev/RRE regulation pathway (Lesnik et al., Med. Res. Rev. 22(6), 617-636, 2002), the antibodies and fragments described herein (which were originally generated against HIV-1 Rev) might be useful in inhibiting polymerization of Rev from lentiviruses other than HIV-1. Alternatively, the provision of SJS-R1 herein enables the identification of similar antibodies specific for the N-terminal domain of the Rev of other lentiviruses. Which Rev variants are likely to bind to SJS-R1 scFv/Fab is related to the residues on Rev making contact (epitope) with the antibody paratope. Table 1 lists the residues of the Rev epitope that are engaged by SJS-R1, all of which are located in the N-terminal region (residues 1-69) of Rev. Rev variants that have different residues at one or more of the positions corresponding to contact residues (listed in Table 1) are likely to have modified binding to a SJS-R1-based antibody molecule. Rev variants that differ in the C-terminal portion of the protein compared to the sequence of the HIV-1 Rev used to elicit SJS-R1 (that is, GenBank Accession No. P04616) are less likely to have reduced binding to SJS-R1 based antibody molecules.

Sample: A biological specimen containing genomic DNA, RNA, protein, or combinations thereof, oral fluid, saliva, sputum, tissue biopsy (such as skin tissue), surgical specimen, and autopsy material.

Specific hybridization: Specific hybridization refers to the binding, duplexing, or hybridizing of a molecule only or substantially only to a particular nucleotide sequence when that sequence is present in a complex mixture (e.g. total cellular DNA or RNA). Specific hybridization may also occur under conditions of varying stringency.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. (In: Molecular Cloning: A Laboratory Manual, Cold Spring Harbor, N.Y., 1989 ch. 9 and 11). By way of illustration only, a hybridization experiment may be performed by hybridization of a DNA molecule to a target DNA molecule which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989).

Stringent conditions may be defined as those under which DNA molecules with more than 25%, 15%, 10%, 6% or 2% sequence variation (also termed “mismatch”) will not hybridize. Stringent conditions are sequence dependent and are different in different circumstances. Longer sequences hybridize specifically at higher temperatures. Generally, stringent conditions are selected to be about 5° C. lower than the thermal melting point Tₘ for the specific sequence at a defined ionic strength and pH. An example of stringent conditions is a salt concentration of at least about 0.01 to 1.0 M Na ion concentration (or other salts) at pH 7.0 to 8.3 and a temperature of at least about 30° C. for short probes (e.g. 10 to 50 nucleotides). Stringent conditions can also be achieved with the addition of destabilizing agents such as formamide. For example, conditions of 5×SSPE (750 mM NaCl, 50 mM Na Phosphate, 5 mM EDTA, pH 7.4) and a temperature of 25-30° C. are suitable for allele-specific probe hybridizations.

The following is an exemplary set of hybridization conditions and is not meant to be limiting:

Very High Stringency (detects sequences that share 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share 80% identity or greater)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share greater than 50% identity)

Hybridization: 6×SSC at RT to 55° C. for 16-20 hours

Wash at least twice: 2×-3×SSC at RT to 55° C. for 20-30 minutes each.

A perfectly matched probe has a sequence perfectly complementary to a particular target sequence. The test probe is typically perfectly complementary to a portion (subsequence) of the target sequence. The term “mismatch probe” refers to probes whose sequence is deliberately selected not to be perfectly complementary to a particular target sequence.

Specific binding agent: An agent that binds substantially only to a defined target. Thus a protein-specific binding agent binds substantially only the specified protein. By way of example, as used herein, the term “X-protein specific binding agent” includes anti-X protein antibodies (and functional fragments thereof) and other agents (such as soluble receptors) that bind substantially only to the X protein (where “X” is a specified protein, or in some embodiments a specified domain or form of a protein, such as a particular allelic form of a protein).

Subject: Living multi-cellular vertebrate organisms, a category that includes both human and non-human mammals. In some embodiments, the subject is a human subject.

Symptom and sign: A “symptom” is any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for immunological status or the presence of lesions in a subject with multiple sclerosis, and the presence of joint inflammation and pain in subjects with arthritis.

Therapeutic agent: A chemical compound, small molecule, or other composition, such as an antisense compound, antibody, antigen-recognizing fragment of an antibody, peptide or nucleic acid molecule capable of inducing a desired therapeutic or prophylactic effect when properly administered to a subject. For example, therapeutic agents for melanoma include agents that prevent or inhibit development or metastasis of melanoma.

Therapeutically effective amount: A dose or quantity of an agent, such as an anti-viral agent, sufficient to achieve a desired effect in a subject being treated. In one specific, non-limiting example, a therapeutically effective amount of an anti-viral agent is the amount necessary to inhibit viral replication, or to measurably alter outward signs and/or symptoms of the viral infection, for example by increasing T cell counts in the case of an HIV infection, and/or reducing cachexia or the incidence of opportunistic infections. When administered to a subject, a dosage will generally be used that will achieve target tissue concentrations (for example, in lymphocytes) that has been shown to achieve in vitro inhibition of viral replication

Therapy: The mode of treatment or care of a patient. In some cases, therapy refers to administration of a therapeutic agent.

Treatment: Refers to both prophylactic inhibition of initial disease or syndrome and therapeutic interventions to alter the natural course of a disease process (e.g., infection with HIV) or syndrome (such as AIDS).

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

Virus: A microscopic infectious agent that reproduces inside living cells. A virus consists essentially of a core of a nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. Viruses include, but are not limited to, lentiviruses such as a human immunodeficiency virus (e.g., HIV-1 and HIV-2).

Wild-type: The customary type of a molecule (or cell) before manipulation or mutation, or the functionally active general form. Thus, a wild-type form of a protein is the form of the protein found in a cell before manipulation or mutation, and a wild-type form of a virus is the form of a virus that infects a cell prior to manipulation or mutation.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Described herein is the surprising identification of a unique antibody Fab SJS-R1 (comprising heavy and light chains, respectively SEQ ID NOs: 7 and 6) which shows high affinity binding against HIV-1 Rev, and can now be exploited as direct therapeutics as well as in the development of additional therapeutics to combat HIV-1 infection and the development of AIDS. Representative methods of making and using SJS-R1 Fab and SJS-R1 scFv, as well as other related and derived anti-Rev antibodies and antibody fragments, are described.

Provided in one embodiment is an anti-Rev antibody or a fragment thereof which maintains binding activity to HIV-1 Rev, which includes a V_{H} region and a V_{L} region. In various examples of this embodiment, the V_{H} region has a framework and comprises three CDRs: a first CDR comprising the amino acid sequence GFWLNW (positions 31-36 of SEQ ID NO: 2); a second CDR comprising the amino acid sequence AIYRGSGSEWYASWAKG (positions 50-66 of SEQ ID NO: 2); and a third CDR comprising the amino acid sequence AADTTDNGYFTI (positions 95-106 of SEQ ID NO: 2); or the V_{H} region has a sequence at least 90% identical to SEQ ID NO: 2. In various examples of this embodiment, the V_{L} region has a framework and comprises three CDRs: a first CDR comprising the amino acid sequence QASQSISSWLS (positions 25-35 of SEQ ID NO: 4); a second CDR comprising the amino acid sequence DASNLAS (positions 51-57 of SEQ ID NO: 4); and a third CDR sequence comprising the amino acid sequence LGGYPAASYRTA (positions 90-101 of SEQ ID NO: 4); or the V_{L} region has a sequence at least 90% identical to SEQ ID NO: 4.

In some examples of this embodiment, the anti-Rev antibody or a fragment thereof which maintains binding activity to HIV-1 Rev comprises a V_{H} region with a framework and comprising a first CDR comprising the amino acid sequence GFWLNW (positions 31-36 of SEQ ID NO: 2); a second CDR comprising the amino acid sequence AIYRGSGSEWYASWAKG (positions 50-66 of SEQ ID NO: 2); and a third CDR comprising the amino acid sequence AADTTDNGYFTI (positions 95-106 of SEQ ID NO: 2); and a V_{L} region with a framework and comprising a first CDR comprising the amino acid sequence QASQSISSWLS (positions 25-35 of SEQ ID NO: 4); a second CDR comprising the amino acid sequence DASNLAS (positions 51-57 of SEQ ID NO: 4); and a third CDR sequence comprising the amino acid sequence LGGYPAASYRTA (positions 90-101 of SEQ ID NO: 4). In other examples, the anti-Rev antibody or a fragment thereof which maintains binding activity to HIV-1 Rev comprises a V_{H} region with a framework and comprising a first CDR comprising the amino acid sequence GFWLNW (positions 31-36 of SEQ ID NO: 2); a second CDR comprising the amino acid sequence AIYRGSGSEWYASWAKG (positions 50-66 of SEQ ID NO: 2); and a third CDR comprising the amino acid sequence AADTTDNGYFTI (positions 95-106 of SEQ ID NO: 2); and a V_{L} region having a sequence at least 90% identical to SEQ ID NO: 4. In yet other examples of this embodiment, the anti-Rev antibody or a fragment thereof which maintains binding activity to HIV-1 Rev comprises a V_{H} region having a sequence at least 90% identical to SEQ ID NO: 2; and a V_{L} region with a framework and comprising a first CDR comprising the amino acid sequence QASQSISSWLS (positions 25-35 of SEQ ID NO: 4); a second CDR comprising the amino acid sequence DASNLAS (positions 51-57 of SEQ ID NO: 4); and a third CDR sequence comprising the amino acid sequence LGGYPAASYRTA (positions 90-101 of SEQ ID NO: 4). In other examples, the anti-Rev antibody or a fragment thereof which maintains binding activity to HIV-1 Rev comprises a V_{H} region having a sequence at least 90% identical to SEQ ID NO: 2; and a V_{L} region having a sequence at least 90% identical to SEQ ID NO: 4.

In any of the various embodiments of the anti-Rev antibody or fragment thereof which maintains binding activity to HIV-1 Rev, the V_{L} region may optionally be a Vκ region.

In some instances, the anti-Rev antibody or fragment comprises a framework of the V_{H} region that is at least 90% or more identical to the framework of SEQ ID NO: 2, and the framework of the V_{κ} region is at least 90% or more identical to the framework of SEQ ID NO: 4. In yet other instances, the anti-Rev antibody or fragment thereof comprises the framework of the V_{H} region is 95% or more identical to the framework of SEQ ID NO: 2, and the framework of the Vκ region is at least 95% or more identical to the framework of SEQ ID NO: 4.

Also provided are anti-Rev antibodies or fragments thereof, wherein the V_{H} region comprises the sequence shown in positions 1-117 of SEQ ID NO: 2, and the Vκ region comprises the sequence shown in positions 2-111 of SEQ ID NO: 4.

In various of the provided embodiments, the anti-Rev antibody fragment is an Fab fragment, an (Fab′)₂, an Fv fragment, or an single chain Fv fragment (scFv), an scFv-Fc, an IgG, or another bivalent antibody format or transbody. Optionally, in any of the provided embodiments, the anti-Rev antibody or Rev-binding fragment may be partially or fully humanized.

One specific provided anti-Rev Fab fragment is Fab SJS-R1. Another is scFv SJS-R1.

Yet another embodiment is an isolated antibody or antibody fragment that binds the same epitope as does the antibody or fragment of described above; for instance, that binds the same epitope as does Fab SJS-R1 or scFv SJS-R1.

Optionally, any of the provided anti-Rev antibodies or Rev-binding antibody fragments or derivatives may be labeled, for instance with a radionuclide, fluorophore, coloring, enzyme, enzymatic substrate, enzymatic factor, enzymatic inhibitor or ligand.

Pharmaceutical compositions comprising one or more of these anti-Rev antibodies or fragments thereof which bind HIV-1 Rev are also provided. By way of example such pharmaceutical compositions may be formulated for use in prophylactic and/or therapeutic treatment to prevent or reduce HIV infection or a symptom of AIDS. Optionally, any of the provided pharmaceutical compositions may further comprise at least one additional therapeutic agent.

Additional embodiments provide isolated polynucleotides encoding the V_{H} or V_{L} region (or both) of one of the anti-Rev antibodies or fragments thereof described herein. Exemplars of such isolated polynucleotides comprise the sequence shown in SEQ ID NO: 1, SEQ ID NO: 3, or both, or SEQ ID NO: 5 or SEQ ID NO: 8, for instance. Also provided are vectors which comprise at least one such isolated polynucleotide, as well as isolated recombinant host cells (e.g., prokaryotic cells or cells of an immortalized eukaryotic cell line) expressing such polynucleotides.

Methods of using the described anti-Rev antibodies and fragments and derivatives are also described. One such method is a method of inhibiting or preventing or reversing multimerization/polymerization of Rev, comprising contacting Rev protein with the antibody or antibody described herein, thereby preventing or reducing polymerization of Rev. Optionally, such method may take place in a cell, for instance a mammalian cell infected with a lentivirus.

Another provided method is a method for preventing or inhibiting replication of a lentivirus in a cell, comprising contacting a cell infected with the lentivirus with the anti-Rev antibody or fragment thereof provided herein, thereby preventing or inhibiting replication of the lentivirus in the cell.

Yet another method provides for reducing infectivity or replication of a lentivirus, which method comprises contacting the lentivirus with the anti-Rev antibody or fragment thereof described herein, thereby reducing infectivity or replication of the lentivirus.

In another embodiment, there is provided a method of inhibiting Rev function in a cell infected with a lentivirus, comprising contacting the cell with the anti-Rev antibody or fragment thereof described herein, thereby inhibiting Rev function in the cell.

A method of treating a disease or symptom associated with Rev expression or activity in an animal is also provided, which method comprises administering to the animal with said disease or symptom a therapeutically effective amount of the anti-Rev antibody or fragment described herein, thereby treating the disease or symptom. Optionally, the subject is infected with a lentivirus.

In several of the provided methods, the lentivirus may be HIV-1, HIV-2, SIV, FIV or another lentivirus that expresses Rev. For instance, in some instances the lentivirus is a human lentivirus.

Yet another embodiment is an article of manufacture comprising the anti-Rev antibody or fragment thereof described herein, for the treatment of an HIV infection or AIDS. Also provided are kits which comprise at least one anti-Rev antibody or fragment thereof provided herein for the treatment of an HIV infection or AIDS.

Use of the anti-Rev antibodies or fragments thereof described herein can also be used to detect HIV or HIV infection, to diagnosis HIV infection, AIDS or ARC, or to monitor disease progression or recovery or treatment.

### IV. Identification and Characterization of SJS-R1 Fab and scFv

Described herein (Example 1) is the production and characterization of SJS-R1, a chimeric rabbit/human anti-Rev Fab that was selected by phage display, expressed in a bacterial secretion system, and purified from the media. The Fab readily solubilized polymeric Rev. The Fab/Rev complex was purified by metal ion affinity chromatography and characterized by analytical ultracentrifugation which demonstrated monodispersity and indicated a 1:1 molar stoichiometry. The Fab binds to the N-terminal domain of Rev with very high affinity (estimated at ˜40 μM), as determined by surface plasmon resonance, to a conformational epitope in the N-terminal half of Rev. The corresponding single chain antibody (scFv) was also prepared.

The following table illustrates reported (Cole et al., Biochem. 32, 11769-11775, 1993; Pond et al., Proc. Natl. Acad. Sci. U.S.A. 106, 1404-1408, 2009; Stahl et al., J. Mol. Biol. 397, 697-708, 2010) binding affinities of Rev interactions that are relevant for its biological function. The binding of SJS-R1 Fab to Rev is significantly stronger than any of the other binding affinities.

| | | K_{d} |
|---|---|---|
| Rev interactions | Features | (nM) |
| Rev + Rev = (Rev)ₙ | unlimited isodesmic | 100 |
| | (polymers formed) | |
| Rev + RRE = (Rev-RRE) | initial high affinity | 0.3 |
| | stem loop binding | |
| (Rev-RRE) + Rev = [RRE (Rev)ₙ] | n > 4 (12) | 0.8 |
| | required for activity | |
| Rev + Fab = Rev-Fab | 1:1 molar complex | 0.04 |

### VI. Uses of SJS-R1 Fab, scFv, and Related Anti-Rev Binding Molecules

The unique antibody Fab SJS-R1 (comprising heavy and light chains, respectively SEQ ID NOs: 7 and 6) shows high affinity binding against HIV-1 Rev, and can now be exploited as direct therapeutics as well as in the development of additional therapeutics to combat HIV-1 infection and the development of AIDS. Since the function of Rev depends on its ability to assemble as a multiprotein complex on the viral RNA targeting sequence RRE, the exceptionally high affinity of SJS-R1 (which is conferred by the CDRs of this Fab/scFv) can be exploited to disassemble or block assembly of Rev in vitro and in vivo.

The scFv form of SJS-R1 (SEQ ID NO: 10, with or without the hexa-His extension) also tightly binds Rev and disassembles Rev multiprotein complexes. With the provisional of SJS-R1 scFv, methods are now enabled for producing intracellular antibody (intrabody) therapeutics against HIV-1, as well as using SJS-R1 scFv and other scFv's that share at least one CDR with SJS-R1 scFv as therapeutics against HIV-1.

An important feature of the SJS-R1 Fab is that it is in a chimeric rabbit/human form, which allows it to be directly used in humans as a potential therapeutic. The variable heavy and light chain domains are rabbit whereas the constant heavy chain domain C₇₁1 and the constant light chain domain C_{K} are human. This chimeric format could be humanized by replacing the rabbit framework regions with human framework regions as described by e.g., Rader et al., J. Biol. Chem. 275(18), 13668-11676, 2000. When used as an intrabody in scFv format, this humanization may be useful to lower the immunogenicity of the intrabody for use in human subjects. Although Fab and other two-chain antibody formats are generally not used as intrabodies, a therapeutic benefit of the chimeric rabbit/human Fab format may result if it is administered and delivered systemically.

In addition, the 3D crystal structure of the Rev/Fab complex described herein and in DiMattia et al., PNAS 107(13):5810-5814, 2010 (incorporated herein by reference in its entirety) can be used for computer-assisted or other rational design of peptides and small molecules that inhibit the functioning of Rev in infected cells, thereby enabling the development of additional HIV-1 therapeutics including small molecule mimics of the SJS-R1 Fab. Methods of rational design based on structural determinations and biochemical data (such as data presented and incorporated herein) can now be used to identify small compounds (including, but no limited to, peptides) that will have an effect similar to SJS-R1 Fab (or scFv) in inhibiting polymerization or depolymerizing HIV-1 Rev, or in the other uses provided herein. By way of example, methods are provided in Peptide and Protein Design for Biopharmaceutical Applications (Knud Jensen, Ed., Wiley, 2009). A number of articles review computer modeling of potential drug compounds interactive with specific-proteins, such as Rotivinen et al. Acta Pharmaceutical Fennica 97:159-166, 1988; Ripka, New Scientist 54-57, 1988; McKinaly and Rossmann, Annu Rev Pharmacol Toxicol 29:111-122, 1989; Perry and Davies, OSAR: Quantitative Structure-Activity Relationships in Drug Design pp. 189-193, 1989 (Alan R. Liss, Inc.); Lewis and Dean, Proc R Soc Lond 236:125-140 and 141-162, 1989. Representative molecular modeling systems are the CHARMm and QUANTA programs (Polygen Corporation, Waltham, Mass.). CHARMm performs energy minimization and molecular dynamics functions. QUANTA performs construction, graphic modeling and analysis of molecular structure. QUANTA allows interactive construction, modification, visualization, and analysis of the behavior of molecules with each other. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

The binding affinities of SJS-R1 Fab and scFv can be further characterized using surface plasmon resonance (Biacore) and titration calorimetry, for instance. The resultant thermodynamic data is then used in conjunction with high resolution structural data of both Rev-antibody fragment complexes and the antibodies alone to even more finely map out the binding surfaces and contacts. This information will enhance the process of designing both higher affinity binding reagents as well as peptide-related and possibly other small molecule inhibitors of Rev polymerization/multimerization activity.

The anti-rev antibodies and binding fragments described herein have numerous in vitro and in vivo diagnostic and therapeutic utilities involving the detection, diagnosis and treatment of HIV-1 (or another lentivirus) infection and related symptoms and disorders. For example, these molecules can be administered to cells in culture, in vitro or ex vivo, or to human subjects, e.g., in vivo, to control viral infection or replication; to treat a disease or symptom associated with Rev expression or activity; to prevent or reverse polymerization of Rev; to prevent or inhibit replication of a lentivirus (such as HIV); to reduce infectivity or replication of a lentivirus (such as HIV); and/or to inhibit Rev function in a cell in the subject infected with a lentivirus (such as HIV); or more generally to treat, prevent and to diagnose HIV-1 infection, AIDS, or ARC or a symptom thereof. Representative subjects are human and include individuals who have been exposed to, are at risk for exposure to, or are infected with HIV-1 virus disorders, including subjects with active HIV-1 infection, clinical AIDS or ARC, or related symptoms or disorders.

Given that the SJS-R1 Fab and scFV provided herein both prevents and reverses assembly of HIV-1 Rev into multiprotein complexes, another embodiment is a method of preventing or reversing polymerization or assembly of Rev in a cell, either in vitro, ex vivo, or in vivo (for instance, in a subject), comprising administering to the cell (or a subject comprising the cell) the anti-Rev antibody or fragment thereof of the invention (e.g., SJS-R1 Fab, SJS-R1 scFv, a Rev-binding polypeptide comprising one or more CDRs thereof, or a nucleic acid molecule encoding such a protein) in an amount effective to prevent or reverse polymerization or assemble of HIV-1 Rev in the cell.

In another embodiment, the anti-Rev antibodies/fragments are used to detect the presence or level of HIV-1 in a sample, or to detect the presence or levels of cells which contain HIV-1, which can then be linked to certain disease symptoms such as the progression or remission of HIV-1 infection. This can be achieved for instance by contacting a sample and a control sample with the anti-Rev antibody/fragment under conditions that enable formation of a complex between the antibody/fragment and Rev. Any complexes formed between the antibody/fragment and Rev are detected and compared in the sample and the control.

Alternatively, the antibodies/fragments can be used to inhibit or block HIV-1 Rev function in a cell which, in turn, can be linked to the prevention, reversal or amelioration of certain disease symptoms, such as one or more symptoms of HIV-1 infection, AIDS, or ARC.

In light of the highly specific binding affinity of the SJS-R1 Fab and scFv antibodies for HIV-1 Rev, these antibody molecules can be used to specifically detect HIV-1 Rev expression and, moreover, can be used to purify HIV-1 Rev via immunoaffinity purification.

In one embodiment, the antibody that specifically binds HIV-1 Rev, or an antigen binding fragment thereof is fully human. Examples of framework sequences that can be used include the amino acid framework sequences of the heavy and light chains disclosed in PCT Publication No. WO 2006/074071 (see, for example, SEQ ID NOs: 1-16 therein).

Antibody fragments are encompassed by the present disclosure, such as Fab (for instance, SJS-R1 Fab), F(ab′)₂, and Fv which include a heavy chain and light chain variable region and which share one or more of the CDRs of SJS-R1. These antibody fragments retain the ability to specifically bind with the HIV-1 Rev antigen. Fragments of antibodies include scFv, diabodies (scFv dimers), minibodies (scFv-CH₃ dimers), and scFv-Fc (scFv-CH₂—CH₃ dimers). The antibodies can be monovalent or divalent. Methods of making these fragments are known in the art (see for example, Harlow and Lane, Using Antibodies: A Laboratory Manual, Cold Spring Harbor Laboratory Press, 1999).

In a further group of embodiments, the antibodies are Fv antibodies, which are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments comprise V_{H} and V_{L} chains connected by a peptide linker. These single-chain antigen binding proteins (scFv) (such as SJS-R1 scFV) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; and Pack et al., Bio/Technology 11:1271, 1993). Dimers of a single chain antibody (scFV₂), are also contemplated.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulthydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Meth. Enzymol. Vol. 1, page 422, Academic Press, 1967; and Coligan et al. Current Protocols in Immunology, John Wiley & Sons, 2002).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. Conservative amino acid substitution tables providing functionally similar amino acids are well known to one of ordinary skill in the art. Examples of conservative substitutions are provided herein.

Also provided are methods of evaluating functionality of anti-Rev antibody molecules (such as for instance variants and fragments described herein); such functional analyses may involve comparing a function (e.g., inhibition or reversal of Rev polymerization, inhibition of HIV infection or replication, inhibition of infectivity, and so forth) of the variant or fragment to the equivalent function of SJS-R1 Fab or scFv, for instance. Such evaluation methods are also useful, for instance, in characterizing in vivo effects of the described anti-Rev antibodies molecules, as well as establishing appropriate therapeutic dosages. Variant anti-Rev antibody molecules can be examined and evaluated for their ability to inhibit Rev multimerization (for instance, HIV-1 Rev, or another Rev from another lentivirus) using methods described herein or disclosed elsewhere (see, e.g., WO 2009/0147196; describing a number of assays). In vivo effects (including reduction of HIV infectivity, reduction of lentivirus replication or RNA splicing, for instance) can also be analyzed, either using techniques described herein or known in the art.

Additional methods to assay for anti-HIV activity include, but are not limited to, a single-cycle infection assay as described in Martin et al. (Nature Biotechnology 21:71-76, 2003). In this assay, the level of viral activity is measured via a selectable marker whose activity is reflective of the amount of viable virus in the sample, and the IC50 is determined. In other assays, acute infection can be monitored in the PM1 cell line or in primary cells (normal PBMC). In this assay, the level of viral activity can be monitored by determining the p24 concentrations using ELISA.

HIV infection does not need to be completely eliminated for the provided composition(s) to be effective. For example, a composition can decrease HIV infection by a desired amount, for example by at least 10%, at least 20%, at least 50%, at least 60%, at least 70%, at least 80%, at least 90%, at least 95%, at least 98%, or even at least 100% (elimination of detectable HIV infected cells), as compared to HIV infection in the absence of the composition. In additional examples, HIV replication can be reduced or inhibited by similar methods. HIV replication does not need to be completely eliminated for the composition to be effective. For example, a composition can decrease HIV replication by a desired amount, for example by at least 10%, at least 20%, at least 50%, at least 60%, at least 70%, at least 80%, at least 90%, at least 95%, at least 98%, or even at least 100% (elimination of detectable HIV), as compared to HIV replication in the absence of the composition. The cell can be in vivo or in vitro.

### V. Methods of Using Anti-Rev Antibodies and Binding Fragments

With the provision herein of SJS-R1 Fab and scFv, and the recognition that these antibody molecules (and related antibody molecules and fragments that include at least one CDR from SJS-R1) have particularly high affinity for HIV-1 Rev, various methods of use are now enabled. One such method involves directly administering one of the anti-Rev therapeutic compound(s), such as SJS-R1, scFv SJS-R1, or another antibody or fragment that binds HIV Rev competitively therewith, to a mammalian subject for control of viral infection or replication, in vivo.

Beneficially, the anti-Rev protein compound is administered in such a way that it interacts with intracellular Rev. Optionally, the anti-Rev antibody or fragment thereof comprises or is attached to a peptide that enhances or encourages (intra)cellular delivery, cell penetration, and/or membrane translocation.

Methods of intracellular delivery of protein and peptide therapeutics are known in the art; see, e.g., Delehanty et al. (Therap Del. 1(3):411-433, 2010), Torchilin (Drug Discov Today: Technol, doi:10.1016/j.ddtec.2009.01.002, 2009) Torchilin (Adv Drug Del Rev. 60(4-5):548-558, 2008), Siprashvili et al. (Mol Thera. 9:721-728, 2004), Torchilin (Proc Natl Acad Sci. 98(5):8786-8791, 2001), Hawiger (Curr Opin Chem Biol. 3(1):89-94, 1999; and Fawell et al. (Proc. Natl. Acad. Sci. USA 91:664-668, 1994).

The described anti-Rev antibody/fragment can also be used to treat a disease or symptom associated with Rev expression or activity in a subject.

Yet another provided method that employs an anti-Rev antibody or fragment there of that maintains binding to HIV-1 Rev is prevention or reversal of polymerization of Rev (that is, assembly of Rev into a multiprotein complex), either in vivo or in vitro.

The described anti-Rev antibodies and fragments described herein may also be used to prevent or inhibit replication of a lentivirus (such as HIV), or to reduce infectivity or replication of a lentivirus (such as HIV), either in vivo or in vitro.

Methods are also provided to inhibit Rev function in a cell in the subject infected with a lentivirus (such as HIV), which methods involve contact the cell of an infected subject (in vivo or ex vivo) with at least one anti-Rev antibody fragment or fragment thereof.

The present disclosure further provides use of anti-Rev antibodies or fragments thereof (such as SJS-R1 Fab or scFv, or a polypeptide comprising one or more of the CDRs thereof) in a medicament and in the preparation of a medicament for the treatment or prevention of a viral infection, such as infection by HIV-1. Also provided by the present disclosure is the SJS-R1 Fab and scFv antibody fragments, and HIV-1 Rev binding derivatives thereof, for use in a method for treating a HIV-1 infection associated disorder, such as AIDS or ARC

A “therapeutically effective amount” of an anti-Rev antibody or fragment described herein preferably results in a decrease in severity of disease symptoms, an increase in frequency and duration of disease symptom-free periods, and/or a prevention of impairment or disability due to the disease affliction. For example, for the treatment of an HIV-1 infection, a “therapeutically effective amount” preferably inhibits viral load by at least about 20%, more preferably by at least about 40%, even more preferably by at least about 60%, and still more preferably by at least about 80% relative to untreated subjects. The ability of a specific anti-Rev antibody molecule to inhibit Rev activity, such as for instance Rev polymerization or HIV infectivity or replication, can be evaluated in an animal model system predictive of efficacy in human viral infection, specifically human HIV-1 infection. The ability of an agent, such as those described, to prevent or decrease infection by HIV, can be assessed in animal models. Several animal models for pathogen infection are known in the art. For example, mouse HIV models are disclosed in Sutton et al. (Res. Initiat Treat. Action, 8:22-24, 2003) and Pincus et al. (AIDS Res. Hum. Retroviruses 19:901-908, 2003)

A therapeutically effective amount of a therapeutic compound can inhibit or reverse or prevent Rev activity in a subject, for instance an amount that can inhibit or prevent HIV-1 infection as measured by any of a number of recognized anti-viral assays, including but not limited to those described herein. One of ordinary skill in the art would be able to determine such amounts based on such factors as the subject's size, the severity of the subject's symptoms, and the particular composition or route of administration selected.

The benefits of treatment with an antibody (or fragment thereof) that specifically binds HIV-1 Rev can be demonstrated in one or more randomized, placebo-controlled, double-blinded, Phase II or III clinical trials and will be statistically significant (e.g., p<0.05).

### VI. Nucleic Acids Encoding Anti-Rev Antibodies and Binding Fragments

Anti-Rev antibodies and fragments described herein can be administered as nucleic acids that express the corresponding polypeptide. For example, a therapeutic polynucleotide is introduced into cells infected with, or at risk of infection with, a lentivirus, resulting in expression of the anti-Rev antibody or fragment within the infected cells. The therapeutic polynucleotide may encode any of the therapeutic anti-Rev antibodies or fragments described, or others having the provided characteristics.

In some instances, the encoded corresponding polypeptide is an intracellular antibody, or intrabody. For a review of uses of intrabodies in therapy against infectious diseases, such as HIV infection, see Rondon & Marasco (Annu. Rev. Microbiol. 51:257-83, 1997). Methods of making and using intrabodies are known in the art. See for instance Marasco et al. (Proc. Nat'l Acad. Sci U.S.A. 90:7889-7893, 1993; describing production of an intrabody based on a scFv directed against HIV gp120). Rev shuttles between nucleus and cytoplasm, so a SJS-R1-based intrabody can be directed to either or both subcellular compartments. This direction could be provided through appropriate signal or other targeting sequences if the intrabody were to be delivered as DNA or RNA and translated in the target cell. If the intrabody were to be delivered as protein (“transbody”) from the outside (that is, systemically), one would need a target-cell specific delivery mechanism whereas direction within the target cell may be less critical. For an example of a transbody that inhibits viral replication, see Poungpair et al. (Bioconjugate Chem. 21(7), 1134-1141, 2010).

Gene therapeutic approaches for treatment of HIV are known to person skilled in the art; representative methods are reviewed by Strayer et al. (Molecular Therapy 5, 33-41, 2002). See also WO 2009/0147196 for additional methods.

The therapeutic polynucleotides include sequences that are degenerate as a result of the degeneracy of the genetic code. Such polynucleotides are operatively linked to a promoter sequence that facilitates the efficient transcription of the inserted genetic sequence of the host. An expression vector used to express a therapeutic polynucleotide typically contains an origin of replication, a promoter, as well as specific gene(s) that allow phenotypic selection of the transformed cells. Vectors suitable for use in the present invention include, but are not limited to the pMSXND expression vector for expression in mammalian cells (Lee and Nathans, J. Biol. Chem. 263:3521, 1988) and retrovirus derived vectors. The DNA segment can be present in the vector operably linked to regulatory elements, for example, a promoter (e.g., immunoglobulin, T7, metallothionein I, or polyhedron promoters).

Delivery of the therapeutic polynucleotide can be achieved using a recombinant expression vector such as a chimeric virus, or a colloidal dispersion system. Delivery can also be achieved with the use of targeted liposomes. Various viral vectors which can be utilized for the introduction of nucleic acids in a cell as taught herein include adenovirus, herpes virus, vaccinia, or an RNA virus suchas a retrovirus. In one embodiment, the vector is a retroviral vector derived from a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). When the subject is a human, a gibbon ape leukemia virus (GaLV) or an amphotropic MoMuLv is utilized.

A number of additional retroviral vectors can incorporate multiple genes. By inserting a sequence encoding an anti-Rev antibody or antibody fragment into the viral vector, along with another gene that encodes the ligand for a receptor on a specific target cell, the vector becomes target specific. Retroviral vectors can be made target-specific by attaching, for example, a sugar, a glycolipid, or a protein. Targeting is also accomplished by using an antibody to target the retroviral vector.

Another targeted delivery system for polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. In one embodiment, the colloidal system is a liposome. RNA, DNA and intact virions can be encapsulated within the aqueous interior and be delivered to cells in a biologically active form (Fraley et al., Trends Biochem. Sci. 6:77, 1981). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

### VII. Expression of Nucleic Acid Molecules and Polypeptides

The expression and purification of proteins, such as an anti-Rev antibody or binding fragment thereof (such as SJS-R1 Fab and scFv) or derived therefrom (for instance, comprising at least one, and up to all six, CDRs of SJS-R1), can be performed using standard laboratory and production techniques. Examples of such methods are discussed or referenced herein, though one of ordinary skill in the art will recognize additional methods that can be employed. After expression, purified proteins have many uses, including for instance functional analyses, structural analyses, diagnostics, and patient therapy. Furthermore, the DNA sequences which encode an anti-Rev antibody or binding fragment, including cDNAs, can be manipulated. Variant or allelic forms of the SJS-R1 anti-Rev antibody or binding fragments thereof may be isolated based upon information contained herein, and may be studied in order to detect altered characteristics such as altered affinity for Rev, altered ability to prevent Rev polymerization, and functional properties of the encoded anti-Rev antibody or binding fragment variant protein (e.g., influence on viral infectivity or other treatment parameters).

Partial or full-length cDNA sequences, which encode for the subject protein (e.g., a V_{L} or V_{H} chain, or both), may be ligated into bacterial expression vectors. Methods for expressing large amounts of protein from a cloned sequence introduced into Escherichia coli (E. coli) or baculovirus/Sf9 cells may be utilized for the purification, localization and functional analysis of proteins. For example, fusion proteins consisting of peptides encoded by a portion of a gene native to the cell in which the protein is expressed (e.g., a E. coli lacZ or trpE gene for bacterial expression) linked to a SJS-R1 Fab protein or domain or fragment thereof may be used in various procedures, for instance to prepare therapeutic compositions comprising one or more of these proteins.

Intact SJS-R1 Fab or scFv protein may also be produced in large amounts for functional studies as well as therapeutic uses. Methods and plasmid vectors for producing engineered proteins, fusion proteins and intact native proteins in culture are well known in the art, and specific methods are described in Sambrook et al. (In Molecular Cloning: A Laboratory Manual, Ch. 17, CSHL, New York, 1989). Such proteins may be made in large amounts, are easy to purify, and can be used for instance for functional assays or as therapeutic molecules. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Suitable methods are presented in Sambrook et al. (In Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (In Molecular Cloning: A Laboratory Manual, Ch. 17, CSHL, New York, 1989). One representative but non-limiting method for expressing SJS-R1 Fab and SJS-R1 scFv is provided in detail in Example 1.

Vector systems suitable for the expression of lacZ fusion genes include the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986).

The DNA sequence can also be transferred from its existing context to other cloning vehicles, such as other plasmids, bacteriophages, cosmids, animal viruses and yeast artificial chromosomes (YACs) (Burke et al., Science 236:806-812, 1987). These vectors may then be introduced into a variety of hosts including somatic cells, and simple or complex organisms, such as bacteria, fungi (Timberlake and Marshall, Science 244:1313-1317, 1989), invertebrates, plants (Gasser and Fraley, Science 244:1293, 1989), and animals (Pursel et al., Science 244:1281-1288, 1989), which cell or organisms are rendered transgenic by the introduction of the heterologous cDNA.

For expression in mammalian cells, a cDNA sequence may be ligated to heterologous promoters, such as the simian virus (SV) 40 promoter in the pSV2 vector (Mulligan and Berg, Proc. Natl. Acad. Sci. USA 78:2072-2076, 1981), and introduced into cells, such as monkey COS-1 cells (Gluzman, Cell 23:175-182, 1981), to achieve transient or long-term expression. The stable integration of the chimeric gene construct may be maintained in mammalian cells by biochemical selection, such as neomycin (Southern and Berg, J. Mol. Appl. Genet. 1:327-341, 1982) and mycophenolic acid (Mulligan and Berg, Proc. Natl. Acad. Sci. USA 78:2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

A cDNA sequence (or portions derived from it) or a mini gene (a cDNA with an intron and its own promoter) may be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., Proc. Natl. Acad. Sci. USA 78:1078-2076, 1981; Gorman et al., Proc. Natl. Acad. Sci USA 78:6777-6781, 1982). The level of expression of the cDNA can be manipulated with this type of vector, either by using promoters that have different activities (for example, the baculovirus pAC373 can express cDNAs at high levels in S. frugiperda cells (Summers and Smith, In Genetically Altered Viruses and the Environment, Fields et al. (Eds.) 22:319-328, CSHL Press, Cold Spring Harbor, N.Y., 1985) or by using vectors that contain promoters amenable to modulation, for example, the glucocorticoid-responsive promoter from the mouse mammary tumor virus (Lee et al., Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-2076, 1981) or neo (Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell Biol. 1:486, 1981) or Epstein-Barr (Sugden et al., Mol. Cell Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, Virology 52:466, 1973) or strontium phosphate (Brash et al., Mol. Cell Biol. 7:2013, 1987), electroporation (Neumann et al., EMBO J 1:841, 1982), lipofection (Felgner et al., Proc. Natl. Acad. Sci USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70; 1987). Alternatively, the cDNA, or fragments thereof, can be introduced by infection with virus vectors. Systems are developed that use, for example, retroviruses (Bernstein et al., Gen. Engr'g 7:235, 1985), adenoviruses (Ahmad et al., J. Virol. 57:267, 1986), or Herpes virus (Spaete et al., Cell 30:295, 1982). Protein (such as anti-Rev binding protein, for instance SJS-R1Fab or scFv) encoding sequences can also be delivered to target cells in vitro via non-infectious systems, for instance liposomes.

Using the above techniques, the expression vectors containing an anti-Rev antibody or binding fragment (such as for instance the SJS-R1 Fab or scFv) or encoding sequence or cDNA, or fragments or variants or mutants thereof, can be introduced into human cells, mammalian cells from other species or non-mammalian cells as desired. The choice of cell is determined by the purpose of the treatment. For example, monkey COS cells (Gluzman, Cell 23:175-182, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

The present disclosure thus encompasses recombinant vectors that comprise all or part of an anti-Rev antibody or binding fragment encoding sequence (for instance, encoding SJS-R1 Fab or scFv) or cDNA sequences, for expression in a suitable host, either alone or as a fusion protein, such as a labeled or otherwise detectable fusion protein. The DNA is operatively linked in the vector to an expression control sequence in the recombinant DNA molecule so that an anti-Rev antibody or binding fragment polypeptide or fusion polypeptide can be expressed, such as for instance SJS-R1 Fab or scFv, or an antibody or binding fragment thereof comprising at least one CDR therefrom. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

The host cell, which may be transfected with the vector of this disclosure, may be selected from the group consisting of E. coli, Pseudomonas, Bacillus subtilis, Bacillus stearothermophilus or other bacilli; other bacteria; yeast; fungi; insect; mouse or other animal; plant hosts; or human tissue cells. Expression of the polypeptides in prokaryotic cells will result in polypeptides that are not glycosylated. Glycosylation of the polypeptides at naturally occurring glycosylation target sites may be achieved by expression of the polypeptides in suitable eukaryotic expression systems, such as mammalian cells.

It is appreciated that for mutant or variant anti-Rev antibody or binding fragment DNA sequences, similar systems are employed to express and produce the mutant product. In addition, fragments of an anti-Rev antibody can be expressed essentially as detailed above, as can fusion proteins comprising all of for instance SJS-R1 Fab of SJS-R1 scFv or a fragment or fragments thereof. Such fragments include individual SJS-R1 protein domains or sub-domains (for instance, one or more CDRs from the light or heavy chain of SJS-R1, with or without the intervening framework sequences), as well as shorter fragments such as peptides. SJS-R1-derived protein fragments having one or more therapeutic properties may be expressed in this manner also, including for instance substantially soluble fragments, or fragments that associate with Rev of domains or sub domains of Rev.

### VIII. Isolation/Purification

In some embodiments, it is beneficial to obtain isolated and purified anti-Rev antibody or binding fragment protein, for instance for use in characterization studies as well as therapeutic uses. One skilled in the art will understand that there are myriad ways to purify recombinant polypeptides, and such typical methods of protein purification may be used to purify the disclosed anti-Rev antibody molecules, including but not limited to SJS-R1 Fab, SJS-R1 scFv, and other antibody proteins that comprise one or more (or all six) of the CDRs thereof. Such methods include, for instance, protein chromatographic methods including ion exchange, gel filtration, HPLC, monoclonal antibody affinity chromatography and isolation of insoluble protein inclusion bodies after over production. In addition, purification affinity-tags, for instance a six-histidine sequence, may be recombinantly fused to the protein and used to facilitate polypeptide purification (e.g., in addition to another functionalizing portion of the fusion, such as a targeting domain or another tag, or a fluorescent protein, peptide, or other marker). A specific proteolytic site, for instance a thrombin-specific digestion site, can be engineered into the protein between the tag and the remainder of the fusion to facilitate removal of the tag after purification, if such removal is desired.

Commercially produced protein expression/purification kits provide tailored protocols for the purification of proteins made using each system. See, for instance, the QJAexpress™ expression system from QIAGEN (Chatsworth, Calif.) and various expression systems provided by INVITROGEN (Carlsbad, Calif.). Where a commercial kit is employed to produce an anti-Rev binding protein, the manufacturer's purification protocol is a preferred protocol for purification of that protein. For instance, proteins expressed with an amino-terminal hexa-histidine tag (such as is described in Example 1) can be purified by binding to nickel-nitrilotriacetic acid (Ni-NTA) metal affinity chromatography matrix (The QIAexpressionist, QIAGEN, 1997).

More generally, the binding specificity of the anti-Rev antibody molecules described herein may be exploited to facilitate specific purification of the proteins. One example method of performing such specific purification would be column chromatography using column resin to which the target molecule, HIV Rev, or an appropriate epitope or fragment or domain of the target molecule (for instance, the N-terminal domain of Rev, such as some or all of residues 1-59 of Rev), has been attached.

In addition to protein expression and purification guidelines provided herein, protein expression/purification kits are produced commercially. See, for instance, the QIAexpress™ expression system from QIAGEN (Chatsworth, Calif.) and various expression systems provided by INVITROGEN (Carlsbad, Calif.). Depending on the details provided by the manufactures, such kits can be used for production and purification of anti-Rev antibodies and related and derived proteins.

In one representative embodiment, purification of the expressed anti-Rev proteins is generally performed in a basic solution (typically around pH 10) containing 6M urea. Folding of the purified protein is then achieved by dialysis against a buffered solution at neutral pH (typically phosphate buffered saline at around pH 7.4).

### IX. Pharmaceutical Preparations and Methods of Administration

Therapeutic compound(s), such as SJS-R1, scFv SJS-R1, or another antibody or fragment that binds HIV Rev competitively therewith, can be administered directly to the mammalian subject for control of viral infection or replication, in vivo; to treat a disease or symptom associated with Rev expression or activity; to prevent or reverse polymerization of Rev; to prevent or inhibit replication of a lentivirus (such as HIV); to reduce infectivity or replication of a lentivirus (such as HIV); and/or to inhibit Rev function in a cell in the subject infected with a lentivirus (such as HIV). Administration is by any of the routes normally used for introducing a compound into ultimate contact with the tissue to be treated. The compounds are administered in any suitable manner, optionally with pharmaceutically acceptable carrier(s). Suitable methods of administering therapeutic compounds, particularly for the control of viral infection or replication, are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985).

Formulations suitable for administration include aqueous and non-aqueous solutions, isotonic sterile solutions (which can contain antioxidants, buffers, bacteriostats, and solutes that render the formulation isotonic), and aqueous and non-aqueous sterile suspensions (which can include suspending agents, solubilizers, thickening agents, stabilizers, and preservatives). By way of example, compositions can be administered, for example, orally. The formulations of compounds can be presented in unit-dose or multi-dose sealed containers, such as ampoules and vials. Solutions and suspensions can be prepared from sterile powders, granules, and tablets of the kind previously described.

The disclosure also contemplates various pharmaceutical and laboratory compositions that inhibit or block lentivirus, such as immunodeficiency virus, infection. The compositions are prepared using an agent that binds to HIV Rev and blocks or inhibits (and/or reverses) its ability to polymerize and mediate viral replication, such as the Fab SJS-R1, the scFv SJS-R1, an antibody or binding fragment thereof that comprises one or more (and up to all six) of the CDRs from SJS-R1, or an antibody or binding fragment that competes therewith for binding to HIV Rev; or a nucleic acid sequence encoding such a protein or peptide.

When the agent is to be used as a pharmaceutical, the agent is placed in a form suitable for therapeutic administration. The agent may, for example, be included in a pharmaceutically acceptable carrier such as excipients and additives or auxiliaries, and administered to a subject. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, nontoxic excipients, including salts, preservatives, buffers and the like, as described, for instance, in Remington's Pharmaceutical Sciences, 15th ed., Easton: Mack Publishing Co., 1405-1412, 1461-1487, 1975, and The National Formulary XIV., 14th ed., Washington: American Pharmaceutical Association, 1975). The pH and exact concentration of the various components of the pharmaceutical composition are adjusted according to routine skills in the art. The concentration of antibody in the formulations can vary widely, e.g., from less than about 0.5%, usually at or at least about 1%, to as much as 15 or 20% by weight, or from 1 mg/mL to 100 mg/mL. The concentration is selected primarily based on fluid volumes, viscosities, etc., in accordance with the particular mode of administration selected. See Goodman and Gilman The Pharmacological Basis for Therapeutics, 7th ed.

Antibodies and fragments for use in the methods disclosed herein can be frozen or lyophilized for storage and reconstituted in a suitable carrier prior to use. One of skill in the art can readily design appropriate lyophilization and reconstitution techniques.

The anti-Rev antibodies and fragments described herein (including for instance intrabody and transbody forms) can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody or fragment of the invention can be administered via a non-parenteral route, such as a topical, epidermal or mucosal route of administration, for example, intranasally, orally, vaginally, rectally, sublingually or topically.

Suitable solid or liquid pharmaceutical preparation forms are, for example, granules, powders, tablets, coated tablets, (micro)capsules, suppositories, syrups, emulsions, suspensions, creams, aerosols, drops or injectable solution in ampoule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science, 249:1527-1533, 1990, which is incorporated herein by reference.

These and other compositions can be used to treat lentiviral infections, such as HIV disease and AIDS, by blocking replication of an immunodeficiency virus. This method involves administering to a subject a therapeutically effective dose of a pharmaceutical composition containing one or more of compounds of the present invention and a pharmaceutically acceptable carrier. The administration of the pharmaceutical composition of the present invention may be accomplished by any means known to the skilled artisan (for example, intravenous, subcutaneous, intra-peritoneal, topical, intra-nasal, or oral administration). The pharmaceutical compositions may be administered locally or systemically.

For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units, and also by multiple administrations of subdivided doses at specific intervals.

Initial dosage ranges can be selected to achieve an inhibitory concentration in target tissues that is similar to in vitro inhibitory tissue concentrations. The dosage should not be so large as to cause adverse side effects, such as unwanted cross-reactions, anaphylactic reactions, and the like. Generally, the dosage will vary with the age, condition, gender, and extent of the disease in the patient and can be determined by one skilled in the art. The dosage can be adjusted for each individual in the event of any contraindications and can be readily ascertained without resort to undue experimentation. In any event, the effectiveness of treatment can be determined by monitoring the viral load of a patient infected with the immunodeficiency virus. The viral load will decrease following administration of an effective agent. By way of example, the amount of virus can be detected using immunoassay detection of an HIV-1 protein, such as Rev; in embodiments of this method, one of the described anti-Rev antibodies or antibody fragments is used. In various embodiments, the level of CD4+ T-cells is also monitored in the patient. Alternatively, viral load, rate of viral replication, or other biometric measures can be monitored.

In treating a patient in accordance with the methods provided herein, a physician may administer a therapeutic compound (e.g., an anti-Rev antibody or binding fragment thereof, particularly one that shares at alt 85% sequence identity with or otherwise binds at the same epitope as SJS-R1 Fab) immediately and continue administration indefinitely, as needed. Upon HIV infection or exposure, even though the patient does not have symptoms of disease, administration of the compounds may be started before symptoms appear, and treatment may be continued indefinitely to prevent or delay the onset of disease.

The dose administered to a patient, in the context of the present disclosure, should be sufficient to result in a beneficial response in the subject over time. The dose will be determined by the efficacy of the particular therapeutic compound employed and the condition of the subject, as well as the body weight or surface area or volume of the area to be treated. The size of the dose also may be influenced by the existence, nature, and extent of any adverse side-effects that accompany the administration of a particular compound in a particular subject.

In some examples, the pharmaceutical composition may be administered by any means that achieve their intended purpose. Amounts and regimens for the administration of the described anti-Rev antibody or fragment that specifically binds Rev will be determined by the attending clinician.

In determining the effective amounts of the therapeutic compound to be administered, a physician may evaluate circulating plasma levels of the compound, associated toxicities, and the production of antibodies to the compound or any degradation products thereof. In general, the dose equivalent of a therapeutic compound as provided herein is from about 1 ng/kg to 100 mg/kg, or alternatively 0.1 to about 100,000 μg/kg, for a typical subject.

Effective dosages (e.g., therapeutically effective amounts) of an anti-Rev antibody or binding fragment as provided herein range from about 0.001 to about 30 mg/kg body weight, such as from about 0.01 to about 25 mg/kg body weight, for example from about 0.4 to about 20.0 mg/kg body weight. The amount of the antibody/fragment that specifically binds HIV-1 Rev can vary according to the size of the individual to whom the therapy is being administered, as well as the characteristics of the disorder being treated. In exemplary treatments, about 1 mg/day, about 5 mg/day, about 10 mg/day, about 20 mg/day, about 50 mg/day, about 75 mg/day, about 100 mg/day, about 150 mg/day, about 200 mg/day, about 250 mg/day, about 400 mg/day, about 500 mg/day, about 800 mg/day, about 1000 mg/day, about 1600 mg/day or about 2000 mg/day is administered. The doses may also be administered based on weight of the patient, e.g., at a dose of 0.01 to 50 mg/kg. In a related embodiment, the antibody or fragment that specifically binds HIV-1 Rev can be administered in a dose range of 0.015 to 30 mg/kg. In an additional embodiment, the antibody that specifically binds HIV-1 Rev is administered in a dose of about 0.015, about 0.05, about 0.15, about 0.5, about 1.5, about 5, about 15 or about 30 mg/kg. Other dosages can be used; factors influencing dosage include, but are not limited to, the severity of the disease, previous treatment approaches, overall health of the patient, other diseases present, etc. One of skill in the art can readily determine a suitable dosage that falls within the ranges, or if necessary, outside of the ranges.

For administration, compounds identified by the methods described n can be administered at a rate determined by the LD₅₀ of the therapeutic compound, and the side effects of the compound at various concentrations, as applied to the mass and overall health of the subject. Administration can be accomplished via single or divided doses.

A therapeutically effective dose is the quantity of a compound according to the disclosure necessary to prevent, to cure or at least partially ameliorate the symptoms of a disease and its complications or to decrease the ability of an immunodeficiency virus to infect or replicate in a cell. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman and Gilman: the Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference. Effectiveness of the dosage can be monitored by any method (e.g., CD4+ count, viral load).

The pharmaceutical compositions of the disclosure, including antibodies, antibody fragments and derivatives, peptides, peptidomimetics, etc., are useful for treating subjects either having or at risk of having an immunodeficiency virus (e.g., HIV) related disorder, such as AIDS or ARC. For example, the compositions are useful for humans at risk for HIV infection, such as after rape or postcoitally. Application of the compounds is also useful to prevent maternal-fetal transmission of HIV. A “prophylactically effective” amount of an agent, for example, refers to that amount that is capable of measurable inhibiting HIV replication and/or infection.

The anti-Rev antibody or binding fragment (such as for instance SJS-R1 Fab, SJS-R1 scFv, or another antibody fragment or derivative comprising one or more of the CDRs of SJS-R1) can also be administered in combination with one or more other drugs useful in the treatment, prevention, or amelioration of viral disease or an associated symptom. For example, the compounds of this invention may be administered, whether before or after exposure to the virus, in combination with effective doses of other anti-viral agents, immunomodulators, anti-infectives, or vaccines, for instance. Administration of the active agents may be either concurrent or sequential administration. For instance, the anti-Rev antibody or Rev-binding fragment thereof that comprises at least one CDR from SJS-R1 may be provided to the subject prior to, subsequent to, or concurrently with one or more conventional antiviral agents.

In one embodiment, a combination treatment uses (in addition to the anti-Rev molecule(s)) at least one additional therapeutic agent for the treatment of a retroviral disease or associated symptom. Combination treatments may include one or more anti-viral agents, broad categories of which include non-nucleoside reverse transcriptase inhibitors (NNRTIs), nucleoside reverse transcriptase inhibitors, HIV-1 protease inhibitors, and viral entry inhibitors, and combinations of two or more thereof. Representative NNRTI compounds include efavirenz, UC-781, HBY 097, nevirapine (11-cyclopropyl-5,11,-dihydro-4-methyl-6H-dipyrido[3,2-b:2′3′-][1,4]diazepin-6-one), delavirdine ((Rescriptor™; Pharmacia Upjohn) (piperazine, 1-[3-[(1-methyl-ethyl)amino]-2-pyridinyl]-4-[[5-[(methylsulfonyl)amino]-1H-indol-2-yl]carbonyl]-, monomethanesulfonate), SJ-3366 (1-(3-cyclopenten-1-yl)methyl-6-(3,5-dimethylbenzoyl)-5-ethyl-2,4-pyrimidinedione), MKC-442 (6-benzyl-1-(ethoxymethyl)-5-isopropyluracil), GW420867x (S-3 ethyl-6-fluoro-4-isopropoxycarbonyl-3,4-dihydro-quinoxalin-2(1H)-one; Glaxo), and HI-443 (N′-[2-(2-thiophene)ethyl]-N′-[2-(5-bromopyridyl)]-thiourea). Representative nucleoside reverse transcriptase inhibitors include but are not limited to abacavir (Ziagen™, GlaxoSmithKline) ((1S,cis)-4-[2-amino-6-(cyclopropylamino)-9H-purin-9-yl]-2-cyclopentene-1-methanol sulfate (salt)), lamivudine (Epivir™, GlaxoSmthKline) ((2R,cis)-4-amino-1-(2-hydroxymethyl-1,3-oxathiolan-5-yl)-(1H)-pyrimidin-2-one), zidovudine (Retrovir™; GlaxoSmithKline) (3′ azido-3′-deoxythymidine), stavudine (Zerit; Bristol-Myers Squibb) (2′,3′-didehydro-3′ deoxythymidine), zacitabine (Hivid™; Roche Laboratories) (4-amino-1-beta-D2′,3′-dideoxyribofuranosyl-2-(1H)-pyrimidone), and didanosine. Representative HIV-1 protease inhibitors include lopinavir (1S-[1R*,(R*),3R*,4R*]]-N-4-[[(2,6-dimethylphenoxy)acetyl]amino]-3-hydroxy-5-phenyl-1-(phenylmethyl)pentyl]tetrahydro-alpha-(1-methylethyl)-2-oxol(2H)-pyrimidineacetamide), saquinavir (N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-(3S)-carboxamide), nelfinavir mesylate ([3S-[2(2S*,3S*),3a,4β,8aβ]]-N-(1,1-dimethylethyl)decahydro-2[2-hydroxy-3-[(3-hydroxy-2-methylbenzoyl)amino]-4-(phenylthio)butyl]-3-isoquinolinecarboxamide mono-methane sulfonate), indinavir sulfate (([1(1S,2R),5(S))]-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythropentonamide sulfate (1:1) salt), amprenavir ((3S)-tetrahydro-3-furyl N-[(1S,2R)-3-(4-amino-N-isobutylbenzenesulfonamido)-1-benzyl-2-hydroxypropyl]carbamate), and ritonavir ((10-Hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid,5-thiazolylmethyl ester, [5S-(5R*,8R*,10R*,11R*)]). Representative HIV-1 fusion or viral entry inhibitors include PRO542 (Progenics Pharmaceuticals, Inc., Tarrytown, N.Y.), T-20 (Trimeric, Inc., Durham, N.C.) (U.S. Pat. Nos. 5,464,933; 6,133,418; and 6,020,459), and T-1249 (U.S. Pat. Nos. 6,345,568 and 6,258,782). Additional examples of antiviral drugs that can be used in combination therapies include: AL-721 (from Ethigen of Los Angeles, Calif.), recombinant human interferon beta (from Triton Biosciences of Alameda, Calif.), Acemannan (from Carrington Labs of Irving, Tex.), gangiclovir (from Syntex of Palo alto, CA), didehydrodeoxythymidine or d4T (from Bristol-Myers-Squibb), EL10 (from Elan Corp. of Gainesville, Ga.), dideoxycytidine or ddC (from Hoffman-LaRoche), Novapren (from Novaferon labs, Inc. of Akron, Ohio), ribavirin (from Viratek of Costa Mesa, Calif.), alpha interferon and acyclovir (from Burroughs Wellcome), 3TC (from Glaxo Wellcome).

Examples of representative anti-infective agents used in the treatment of HIV, and that could be used in combination with the composition, include clindamycin with primaquine (from Upjohn, for the treatment of pneumocystis pneumonia), fluconazlone (from Pfizer for the treatment of cryptococcal meningitis or candidiasis), nystatin, pentamidine, trimethaprim-sulfamethoxazole, and many others.

Examples of immunomodulators that can be used in combination with the composition are AS-101 (Wyeth-Ayerst Labs.), bropirimine (Upjohn), gamma interferon (Genentech), GM-CSF (Genetics Institute), IL-2 (Cetus or Hoffman-LaRoche), human immune globulin (Cutter Biological), IMREG (from Imreg of New Orleans, La.), SK&F106528, TNF (Genentech), and soluble TNF receptors (Immunex).

“Highly active anti-retroviral therapy” or “HAART” refers to a combination of drugs which, when administered in combination, inhibits a retrovirus from replicating or infecting cells better than any of the drugs individually. In the treatment of HIV, an example of HAART is the administration of 3′ axido-3-deoxy-thymidine (AZT) in combination with other agents. Other examples of HAART regimens include nucleoside analog reverse transcriptase inhibitor drugs, NNRTI drugs, and protease inhibitor drugs. One specific, non-limiting example of HAART is a combination of indinavir and efavirenz (an NNRTI). The details of HAART undergo frequent evolution as new antiviral agents are found. The compositions described herein and/or identified by methods herein described, could be administered in conjunction with (or as part of) HAART.

### X. Methods of HIV Detection, Diagnosis, and Monitoring

Detection of HIV infection using one of the anti-Rev antibodies or binding fragments provided herein is carried out by contacting samples taken from subjects (such as saliva or blood samples) with the anti-Rev antibody or a fragment thereof, and determining the amount of Rev antigen in the sample from the amount of bound anti-Rev antibody. The amount of antibody may be detected by known methods of immunological measurement. For example, immunoprecipitation, immunoagglutination, labeled immunoassay, turbidity immunoassay or the like may be used. In labeled immunoassay, the antibody titer in a sample is represented by the amount of label detected directly with a labeled antibody. Alternatively, the antibody titer may be represented relatively using an antibody of known concentration or known titer as a standard solution. Briefly, a standard solution and a sample are measured with a meter; then, using the resultant value of the standard solution as a standard, the antibody titer in the sample may be expressed relatively. As a labeled immunoassay, any known method such as ELISA, EIA, RIA, FIA (fluoroimmunoassay) or luminescence immunoassay may be used.

By using the high affinity antibody of the invention, it is possible to evaluate the efficacy of AIDS therapeutics (including those described herein) with high sensitivity, as well as to monitor on-going treatment of a subject undergoing anti-HIV treatment. Efficacy evaluation using the high affinity anti-Rev antibody or fragment described herein may be carried out as follows. Drug(s), for instance test drug(s), are administered to AIDS patients or AIDS model animals prepared by transplanting human lymphocytes (SCID-Hu mouse); then, the amounts of HIV (as correlated with the amount of Rev protein) in these bodies or the amounts of immunodeficient virus in model animal bodies are detected with the high affinity anti-Rev antibody. By comparing the resultant amounts, it is possible to evaluate the efficacies of test drugs as an AIDS therapeutic through the amounts of the Rev antigen in bodies. At this time, the antibody of the invention is expected to have sensitivity that may be higher than that of previously employed antibodies.

The high affinity anti-Rev antibody or fragment as described herein may be provided in a form of diagnosis kit for flavivirus infection, for instance for detection or diagnosis of HIV-1 infection. This kit may be used in diagnosis as well as determining or evaluating efficacy of a treatment method described herein, or of another method of treating HIV-1 infection. Further, this kit may also be used as a highly sensitive, rapid and simple kit for checking the presence/absence of HIV infection, for instance in blood transfusion preparations and other biological samples. Such kits comprise an anti-Rev antibody or fragment (such as SJS-R1 Fab or scFv, or an antibody molecule that shares at least one but up to all six CDRs thereof) with our without a labeled, or an immobilizing reagent in which such labeled or unlabeled antibody is fixed. In this context, the labeled antibody means an antibody (or fragment or derivative which still binds HIV-1 Rev) labeled with an enzyme, radioactive isotope, fluorescent compound, chemiluminescent compound, or other readily detectable label.

### XI. Articles of Manufacture and Kits

Another embodiment of the disclosure provides an article of manufacture or a kit comprising an anti-Rev antibody or fragment thereof (e.g., SJS-R1 Fab, SJS-R1 scFv, or another antibody fragment comprising at least one CDR thereof), or comprising a composition comprising such molecules, for use in one of the described methods. The article of manufacture may comprise a container and a label or package insert on or associated with the container. Suitable containers include, for example, bottles, vials or syringes. The containers may be formed from a variety of materials such as glass or plastic. The container holds a composition that may be effective for treating the condition and may have a sterile access port (e.g., the container may be an intravenous solution bag or a vial having a stopper pierceable by a hypodermic injection needle). At least one active agent in the composition may be one of the anti-Rev antibody molecules described herein. The label or package insert may indicate that the composition may be used for treating the condition of choice, such as a viral infection or associated disorder or symptom. In one embodiment, the label or package insert indicates that the composition comprising the anti-Rev antibody or fragment may be used to treat an HIV-1 infection, or AIDS or ARC. In another embodiment, the label or package insert indicates that the composition comprising the anti-Rev antibody or fragment may be used to inhibit or reverse Rev polymerization or assembly into a multiprotein complex. Optionally, the anti-Rev antibody or fragment further comprises a label.

Moreover, the article of manufacture or kit may comprise a first container with a composition contained therein, wherein the composition comprises an anti-Rev antibody or fragment described herein, and a second container with a composition contained therein, wherein the composition in the second container comprises a therapeutic agent other than the anti-Rev antibody or fragment. The article of manufacture in this embodiment may further comprise a package insert indicating that the first and second compositions can be used in combination to treat an HIV-1 infection, or a disease or disorder or symptom associated with an HIV-1 infection. Such therapeutic agent may be any of the “combination” therapy agents described herein, for instance. Optionally, the anti-Rev antibody or fragment further comprises a label.

Optionally, the article of manufacture or kit may comprise further container(s) comprising one or more additional substances that might be useful in carrying out one of the described methods, for instance a pharmaceutically acceptable buffer, or diluent. Optionally, the article of manufacture or kit may also include one or more filters, needles, and/or syringes.

Also within the scope of the present disclosure are kits comprising the SJS-R1 Fab antibody, the SJS-R1 scFv antibody fragment, or another antibody molecule comprising one or more (or all six) of the CDRs from SJS-R1, and instructions for use of that compound or a composition containing it in one of the provided methods. The kit can further contain one or more additional reagents, such as an anti-viral compound.

In addition to the above-described components, the article of manufacture or kit may comprise one or more reagent(s) useful to conduct detection of the anti-Rev antibody molecule. For instance, in an embodiment where the anti-Rev antibody is labeled with an enzyme, the article of manufacture or kit may optionally include enzyme substrate component(s), enzyme substrate solution, enzyme reaction termination solution, or the like.

The following examples are provided to illustrate certain particular features and/or embodiments. These examples should not be construed to limit the disclosure to the particular features or embodiments described.

### EXAMPLES

At least some of the research discussed in the following examples was published in Stahl et al. (J. Mol. Biol. 397, 697-708, 2010) and/or DiMattia et al., Proc. Natl. Acad. Sci. U.S.A. 107, 5810-5814, 2010), both of which are incorporated herein by reference in their entirety.

### Example 1

### Generation and Characterization of a Chimeric Rabbit/Human Fab Specific for HIV-1 Rev

Rev is a key regulatory protein of HIV-1. Its function is to bind to viral transcripts and effect export from the nucleus of unspliced mRNA thereby allowing the synthesis of structural proteins. Despite its evident importance, the structure of Rev has remained unknown, primarily because Rev's proclivity for polymerization and aggregation is an impediment to crystallization. Monoclonal antibody antigen-binding domains (Fabs) have proven useful for the co-crystallization of other refractory proteins.

As described in this Example, a chimeric rabbit/human anti-Rev Fab (SJS-R1) was selected by phage display, expressed in a bacterial secretion system, and purified from the media. The Fab readily solubilized polymeric Rev. The resulting Fab/Rev complex was purified by metal ion affinity chromatography and characterized by analytical ultracentrifugation which demonstrated monodispersity and indicated a 1:1 molar stoichiometry. The Fab binds with very high affinity, as determined by surface plasmon resonance, to a conformational epitope in the N-terminal half of Rev. The complex forms crystals suitable for structure determination. The ability to serve as a crystallization aid is a new application of broad utility for chimeric rabbit/human Fab. The corresponding single chain antibody (scFv) was also prepared.

### Materials and Methods

Preparation of HIV-1 Rev: Rev (clone BH10) was expressed in E. coli and purified as previously described (Wingfield et al., Biochemistry 30, 7527-34, 1991). Protein in 4 M urea was diluted with 6 M urea to a concentration of 69 μg/mL (which is below the polymerization concentration of 80 μg/mL) and then folded by dialysis against 50 mM sodium phosphate (pH 7), 150 mM sodium chloride, 600 mM ammonium sulfate, 1 mM EDTA, 1 mM DTT at 4° C. The Rev was then dialyzed extensively against 50 mM sodium phosphate, pH 7.0, 150 mM sodium chloride. The material was sterile-filtered (0.2 μm) and then snap-frozen in liquid nitrogen in aliquots at 66 μg/mL. A C-terminally truncated form of the protein, Rev^{ΔC60-116}, was also expressed and purified in a similar manner.

Biotinylation of Rev: Rev with a 14-residue biotin ligase substrate domain (Avitag) appended to its C-terminus (Beckett et al., Protein Sci 8, 921-929, 1999) was expressed in E. coli and purified by ion-exchange and gel filtration chromatographies in buffers supplemented with 2 M urea. Biotinylation with biotin ligase (Avidity, LLC) was done according to the manufacturer's protocol. Following the reaction, the protein was gel filtrated on Superdex S200 using 20 mM Tris (pH 7.4) containing 2 M urea. The integrities of the Rev-AviTag and biotinylated proteins were confirmed by mass spectrometry.

Inoculation of rabbits with Rev: All immunization protocols were reviewed and approved by the Animal Care and Use Committees of the NIAID (ASP L1-6) and Spring Valley Laboratories (Sykesville, Md.) where the animals were housed and injected. Two rabbits (1QQ174-1 and 1QQ82-1) homozygous for immunoglobulin allotypes V_{H}a1 and C_{κ}b9 were bled for serum prior to immunization and seven days after each immunization; each rabbit was immunized with 0.5 mL of 66 μg/mL Rev that was stored frozen as 1.2 mL aliquots for injection of two rabbits. Precautions were taken to not warm the protein when it was mixed with an equal volume of Ribi adjuvant and the mixture was placed on ice. One mL was injected per rabbit. Boosts were then given at 3-week intervals using the same amounts of antigen and Ribi adjuvant. After four boosts, when serum titers had stabilized in both rabbits, a final boost was given and 5 or 6 days later the rabbits (1QQ174-1; 1QQ82-1) were euthanized and bone marrow and spleens collected and immediately stored in TRIzol® RNA preparation Reagent (Invitrogen).

Rabbit anti-Rev antibody titers: Antibody titers were monitored by dot blot. Rev was immobilized directly on a 0.45 μm PVDF membrane at a density of 500 ng per dot. Sera from both animals were diluted serially (10-folds) and antibody levels were determined with an anti-rabbit antibody kit (WesternBreeze, Invitrogen).

Selection of anti-Rev chimeric rabbit/human Fab by phage display: Spleen and bone marrow from both rabbits were processed for total RNA preparation and RT-PCR amplification of rabbit V_{κ}, V_{λ}, and V_{H} encoding sequences using established primer combinations and protocols (Rader, Methods Mol Biol 525, 101-28, xiv. 2009). For each rabbit, rabbit V_{L}/human C_{κ}/rabbit V_{H} segments were assembled in one PCR amplification step, digested with SfiI, and cloned into phage display vector pC3C (Hofer et al., J Immunol Methods 318, 75-87, 2007). Electrotransformation of E. coli strain XL1-Blue (XL1-Blue recA1, endA1, gyrA96, thi-1, hsdR17(r_{K}−, m_{K}+), supE44, relA1, lac, [F′, proAB, lacI^{q}ZΔM15::Tn10(tet^{r})]; Stratagene) yielded approximately 2×10⁸ independent transformants.

Based on established protocols (Rader, Methods Mol Biol 525, 101-28, xiv. 2009), the library was selected against biotinylated HIV-1 Rev immobilized on streptavidin-coated microtiter plates (Sigma) at 100 ng/well. After four rounds of panning, 11 out of 12 clones that were analyzed by ELISA (Rader, Methods Mol Biol 525, 101-28, xiv. 2009) revealed strong binding to biotinylated Rev. One clone was negative. AluI restriction analysis showed that all 11 anti-Rev ELISA-positive clones had the same restriction pattern. The negative clone had a different pattern. Alu1 restriction analysis was done on 19 additional clones. Thirteen had the same fingerprint as the 11 original positive clones. The six clones with a different fingerprint were all negative. A fifth round of panning did not yield additional positive clones. Six positive clones (three from the fourth and three from the fifth round of panning) were sequenced as described (Rader, Methods Mol Biol 525, 101-28, xiv. 2009). The sequences were all identical. Surprisingly, all clones had a stop codon (TAG) at the position of the first codon of the V_{H}-C_{H}1 chain (corresponding positions 817 to 819 of SEQ ID NO: 5), which immediately follows the pelB signal sequence in pC3C (as the Fab was obviously being expressed, the host, E. coli strain XL1 Blue, must be suppressing this amber codon through its supE44 genotype).

Expression and purification of anti-Rev Fab and scFv: In order to maximize the expression of the selected anti-Rev Fab, the stop codon was changed, using PCR and the appropriate primers, from TAG (stop) to CAG (glutamine), the customary first codon of the V_{H} variable domain; the resulting sequence is shown in SEQ ID NO: 55. A modified ompA-V_{κ}-C_{κ}-pelB-V_{H}-C_{H}1-polyHis cassette (without HA tag) was transferred from pC3C into E. coli expression vector pET11a (Novagen) between the NdeI-BamHI restriction sites. The anti-Rev Fab V_{κ} and V_{H} sequences were also cloned into pET11a such that scFv versions of them would be expressed joined by the 18-residue linker GGSSRSSSSGGGGSGGGG (positions 112 to 130 of SEQ ID NO: 10, for instance), i.e. ompA-V_{κ}-linker-V_{H}-polyHis (SEQ ID NO: 8, encoding SEQ ID NO: 9 (precursor) and SEQ ID NO: 10 (mature SJS-R1 scFv)). Similar to anti-Rev Fab, these scFv also had a C-terminal polyHis to facilitate purification.

The expression plasmids for Fab or scFv production were transfected into E. coli strain BL21CodonPlusRIL (Stratagene) and the resulting transfectants fermented in a Biostat B 2-L bench-top fermentor (Braun Biotech) using a glycerol carbon source. Cells were grown at 37° C. and induced with IPTG with a typical cell yield of 50-60 g wet weight/L. The fermentation broth was frozen at −80° C., thawed, briefly sonicated and then clarified by centrifugation in a JA-10 rotor at 14,000×g for 30 minutes. The supernatant was added to ˜100 mL of Chelating Sepharose Big Beads resin (GE Healthcare Life Sciences) charged with NiSO₄ and equilibrated with 20 mM sodium phosphate buffer (pH 7.2). The resin was gently mixed for ˜30 minutes, filtered through a Buchner funnel, washed with column buffer containing 1 M urea and then packed into a Ni-Sepharose Fast Flow column (3.5 cm diameter×10 cm length). After washing the column, the protein was eluted using a 10-500 mM imidazole gradient. Pooled fractions were further purified by gel filtration on a Superdex S200 column (2.6 cm diameter×60 cm length) equilibrated in 20 mM sodium phosphate (pH 7.2), 30 mM sodium chloride and 1 M urea. Column fractions were assayed by reducing (plus DTT) and non-reducing (minus DTT) SDS-PAGE. The anti-Rev Fab is formally identified as Fab SJS-R1 (for simplicity hereafter in this example ‘Fab’), and the derived anti-Rev scFv as scFv SJS-R1 (hereafter in this example ‘scFv’).

Preparation of Fab/Rev Immune Complexes: the Fab/Rev Complex was Produced by combining Fab and a 5-fold molar excess of Rev in 1 M Urea. The urea was necessary to prevent the precipitation of Rev. The mixture was dialyzed against PBS, any precipitated protein (usually excess Rev) was removed by centrifugation, and then applied to a Ni-Sepharose Fast Flow column equilibrated in dialysis buffer. The Fab/Rev complex was eluted with imidazole, dialyzed against 20 mM HEPES (pH 8.0) and concentrated to 8-9 mg/mL using an Amicon Ultra-15 10K NMWL centrifugal filter (Millipore). The scFv/Rev complex was produced and purified in a similar manner except that the final dialysis was performed against PBS.

SDS-PAGE and densitometry: Gel electrophoresis was performed using NuPAGE Novex 4-12% polyacrylamide gels (Invitrogen). Gels were scanned and TIF image files analyzed using Kodak Molecular Imaging software V4.0 (Carestream Health, Rochester, N.Y.).

Analytical ultracentrifugation: A Beckman Optima XL-I analytical ultracentrifuge, absorption optics, an An-60 Ti rotor and standard double-sector centerpiece cells were used. Equilibrium measurements were taken at 20° C. and concentration profiles recorded after 16-20 hours at either 12,000-14,500 (Fab/Rev) or 18,000-21,500 (scFv/Rev or scFv) rpm. Baselines were established by over-speeding at 45,000 rpm for 3 hours. Data (the average of five scans collected using a radial step size of 0.001 cm) were analyzed using the standard Optima XL-I data analysis software. Protein partial specific volumes were calculated from the amino acid compositions (Cohn & Edsall, “Proteins, amino acids and peptides,” Van-Nostrand-Reinhold, Princeton, N.J., 1943) and solvent densities were estimated using the public domain software program SEDNTERP (available on the World Wide Web at rasmb.bbri.org/). Sedimentation velocity measurements at 20° C. were taken at 45,000 rpm for 3 hours with data collection at 5-10 minute intervals. Data (radial step size 0.003 cm) was analyzed using the program DCDT+ version 2.2.1 (Philo et al., Anal Biochem 279, 151-63, 2000).

Electron microscopy: Fab and Rev filaments prepared as described previously (Watts et al., J. Struct Biol 121, 41-52, 1998) in cold 50 mM HEPES, 150 mM sodium chloride, 25 mM sodium citrate (pH 7.0) were mixed in equimolar ratio. The mixtures were incubated overnight at 4° C. (conditions under which Rev filament controls remained polymerized). Specimens were applied to 400-mesh carbon-coated copper grids made hydrophilic by air glow discharge and negatively stained with 1% uranyl acetate. Images were recorded with a Philips CM120 electron microscope by CCD at 45,000× magnification.

Surface plasmon resonance: The kinetics of the Fab binding to immobilized Rev was studied by surface plasmon resonance using a Biacore X (GE Healthcare). Rev was immobilized on the surface of a CM5 sensor chip at 1500 RU (1000 RU ˜1 ng bound protein/mm²; Stenberg et al., J Colloid Interface Sci 143, 513-526, 1991) by EDC-NHS coupling chemistry according to the manufacturer's protocol. HBS-EP buffer (GE Healthcare) was used as the running buffer and the analyte Fab (90 μl) in running buffer was passed over immobilized Rev at a flow rate of 30 μL/min. Analyte was injected at concentrations between 0.5-500 nM. For all assays, at least two replicate injections at the same concentrations were employed to calculate the kinetic data. Analysis was done using BIAevaluation software, version 3.1. The interaction was globally fitted to a 1:1 interaction Langmuir model with the association and dissociation phases of the interaction fitted simultaneously. The goodness of fit between the fitted curves and the experimental curves was assessed by visual comparison. The rate constants (kₐ and k_{d}) and the equilibrium constant (K_{d}) were calculated from the best-fit kinetic parameters (BIAevaluation Handbook BR 1002-29, Biacore AB).

Liquid chromatography and mass spectrometry: An HP1100 LC-MS electrospray mass spectrometer (Agilent) coupled to a Zorbax C-3, 2.1 mm diameter×15 cm length column was used. Protein (0.1-0.5 mg/mL) was diluted between 1:20 to 1:50 with either H₂O or 1% formic acid and samples (5 μL) were applied to the column equilibrated in 0.1% formic acid, 5% acetonitrile. The column was washed for 15 minutes with column solvent and then a 35-minute gradient of 5% to 100% acetonitrile in 0.1% formic acid was applied. The flow rate was 0:2 mL/min and gradient eluate was analyzed by mass spectrometry.

Dynamic light scattering: Proteins (1-9 mg/mL) were either centrifuged or filtered prior to analysis and then 10 μL samples were manually pipetted into a 384 well plate (Greiner Bio-One). Measurements were made at 20° C. using a DynaPro™ Plate Reader Plus and the data analyzed using Dynamics V6 software (Wyatt Technology Corporation).

Crystallization and data collection: Purified Fab/Rev in 20 mM HEPES (pH 8.0) at a concentration of 12.9 mg/mL was used. Crystallization trials were at 21° C. in hanging drops containing 200 nL protein and 200 nL precipitant solution equilibrated against 50 μL reservoirs in 96-well plates. Over 1600 reservoir conditions were assayed and only one resulted in crystalline nucleations. Microcrystals of Fab/Rev initially grew in 12%. PEG 6000, 100 mM di-ammonium phosphate (DAP), and 100 mM Tris-HCl (pH 8.5) and were optimized with a screen varying concentration and pH of the initial reservoir components as well as screening of the initial crystallization condition against the 96-well Hampton Research (Laguna Niguel, Calif., USA) additive screen kit. Optimized crystals were grown with Fab/Rev sample at 7.1 mg/mL in 20 mM HEPES, pH 8.0 in 1-2 weeks with 50 mM spermidine added to the drop and in 9-14% PEG 600, 100-200 mM DAP, 100 mM Tris (pH 8.5) and were cryoprotected by a quick pass through reservoir solution supplemented with 25% (v/v) ethylene glycol before flash cryo-cooling in a cold (100 K) stream of nitrogen gas. Diffraction data were recorded from a single Fab/Rev crystal at λ=0.97960 Å at the European Synchrotron Radiation Facility (ESRF) in Grenoble, France. The crystal used for diffraction was grown in 9.5% PEG 6000, 150 mM DAP, 100 mM Tris-HCl (pH 8.5), and 50 mM spermidine. Diffraction data were integrated and scaled using HKL2000 software (HKL Research, Inc.).

Sequence accession numbers: The amino acid sequences for the Fab SJS-R1 L- and H-chains have been deposited in GenBank with accession numbers GU223201 (SEQ ID NO: 4) and GU223202 (SEQ ID NO: 2), respectively.

### Results and Discussion

### Selection of Rev-Specific Antibody Fragments Using Phage Display

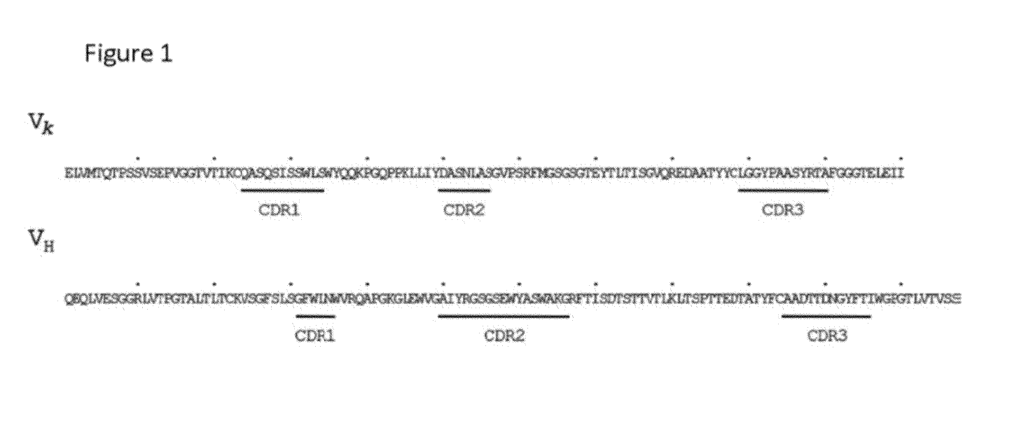

Following immunization with purified recombinant HIV-1 Rev, spleen and bone marrow from two rabbits were collected and processed for total RNA preparation, RT-PCR amplification of rabbit Vₖ, V_{λ}, and VH encoding sequences, Vₖ-Cₖ-V_{H} cassette assembly, and asymmetric SfiI ligation into phage display vector pC3C essentially as described (Rader, Curr Protoc Protein Sci Chapter 6, Unit 6 9, 2009). The resulting library, which consisted of approximately 2×10⁸ independently transformed chimeric rabbit/human Fab clones, was screened by phage display on recombinant Rev protein that had been selectively biotinylated at the C-terminus and immobilized on streptavidin-coated plates. After four to five rounds of panning, selected clones were subjected to initial characterization by ELISA, DNA fingerprinting, and DNA sequencing. All selected clones were identical. The encoded chimeric rabbit/human Fab was termed SJS-R1. The deduced amino acid sequences of the light and heavy variable domains of SJS-R1 (positions 2-111 of SEQ ID NO: 4 and positions 1-117 of SEQ ID NO: 2, respectively), which are shown in FIG. 1, revealed unique rabbit Vₖ and V_{H} sequences with the highest similarity (85% and 77% amino acid sequence identity, respectively) to b9-allotype-derived rabbit variable domains in GenBank (Popkov et al., J Mol Biol 325, 325-35, 2003).

### Expression and Purification of Antibody Fragments

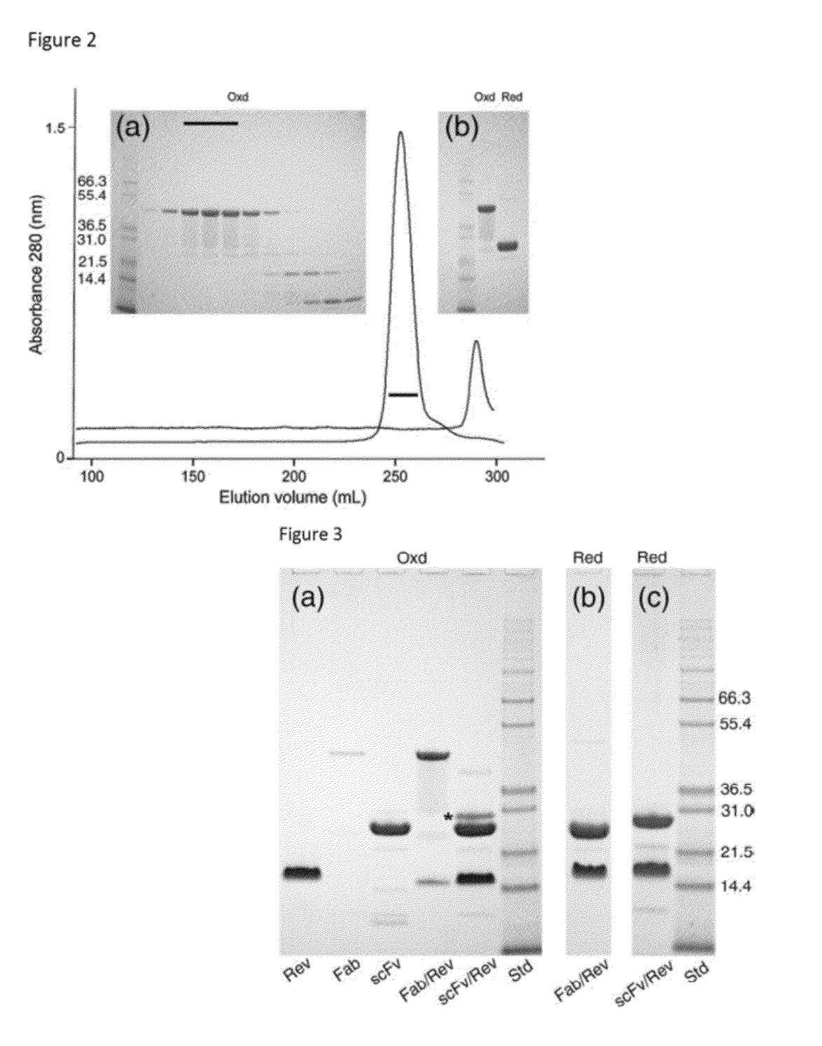

The Fab was expressed in E. coli using an expression cassette with two N-terminal signal sequences pelB and ompA which direct the separate secretion of the Vₖ-Cₖ and V_{H}-C_{H}1 chains into the periplasmic space where enzymic oxidation processes form two intramolecular disulfides per chain and one inter-chain disulfide (Kwong & Rader, Curr Protoc Protein Sci Chapter 6, Unit 6 10, 2009). Although high expression was achieved, most of the Fab protein was retained in the periplasm with only a small amount being secreted into the media. It was found that freezing and thawing of the cells followed by a brief sonication dramatically increased the yield. The His tag on the V_{H}-C_{H}1 chain provided the basis for affinity purification and two cycles of Ni-Sepharose chromatography, one for batch-wise capture and the second using gradient elution, gave good results in terms of yield and purity. When further purification was required, this was carried out using Superdex S200 gel filtration in the presence of 1 M urea to increase Fab solubility (FIG. 2). Using the same expression and purification methods, the corresponding single chain Fab (scFv) was also produced.

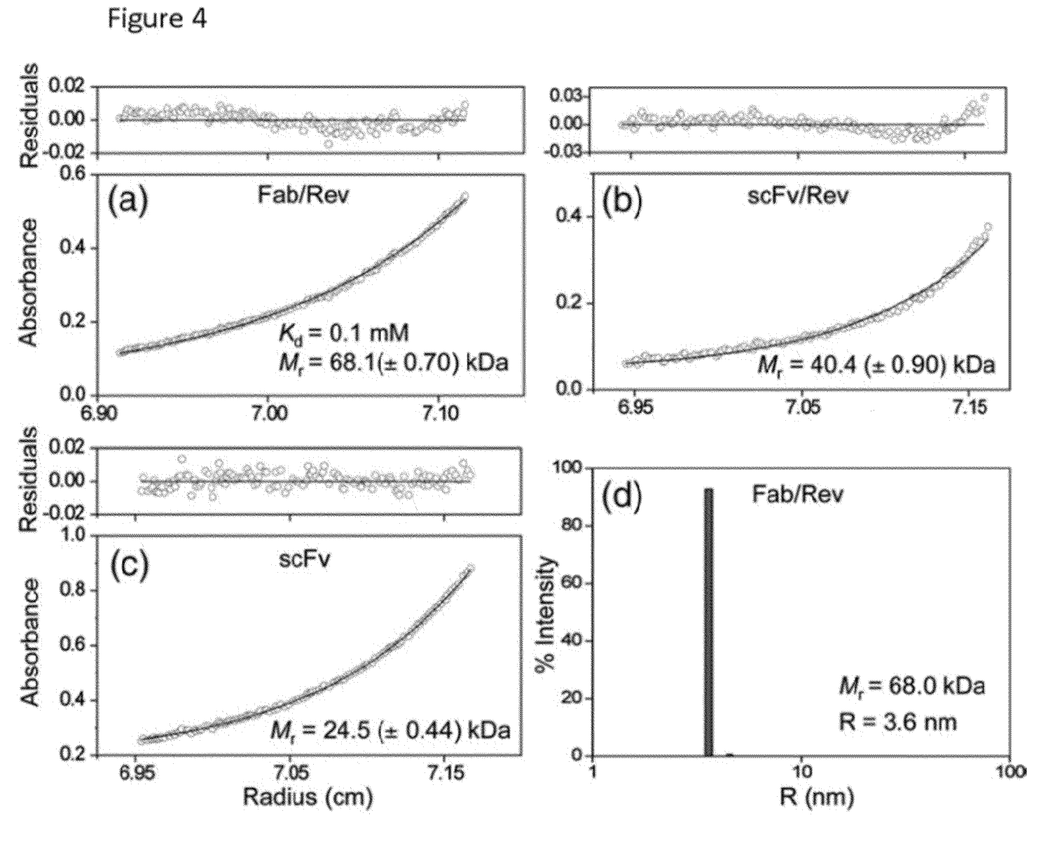

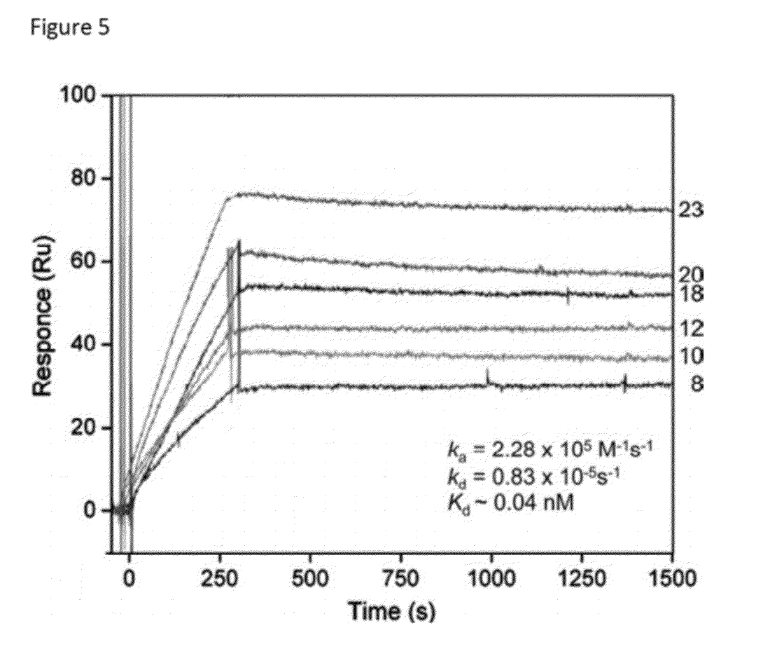

The identities of the antibody fragments were confirmed by mass spectrometry. Reduced Fab gave mass values of 23,464 Da (23,468) and 24,472 Da (24,466) for the light and heavy chains, respectively; and oxidized scFv gave a mass of 26,429 Da (26,430). Values in parentheses are those predicted from the DNA sequences (Note: the Fab heavy chain fragment and scFv include C-terminal His tags). SDS-PAGE of non-reduced Fab (FIG. 2b) and scFv (FIG. 3a, lane scFv) also gave bands with mobilities consistent with the predicted masses. Molecular weights under native conditions were measured by sedimentation equilibrium and both Fab and scFv were found to be monomeric (FIG. 4c). Although the antibody fragments in standard buffers and at neutral pH were well behaved below 1 mg/mL, at higher concentrations there was a tendency towards aggregation and, as mentioned above, the addition of 1 M urea helped maintain solubility without compromising conformational integrity.

### Binding of Fab to Rev

The binding kinetics of the Fab to Rev were measured by surface plasmon resonance with Rev immobilized on the chip and Fab as the analyte (FIG. 5). The binding was characterized by a typical on-rate (kₐ=2.2×10⁵ M^{—1}s⁻¹) but the off-rate (k_{d}=0.8×10⁻⁵s⁻¹) was very low: a low off-rate is characteristic of high affinity (see for example, Drake et al., Anal Biochem 328, 35-43, 2004). With such a low off-rate, and with the technical limitations of the method, the sub-nanomolar affinity value determined (˜40 pM) needs confirmation by another approach but regardless, a high affinity interaction is certainly suggested.

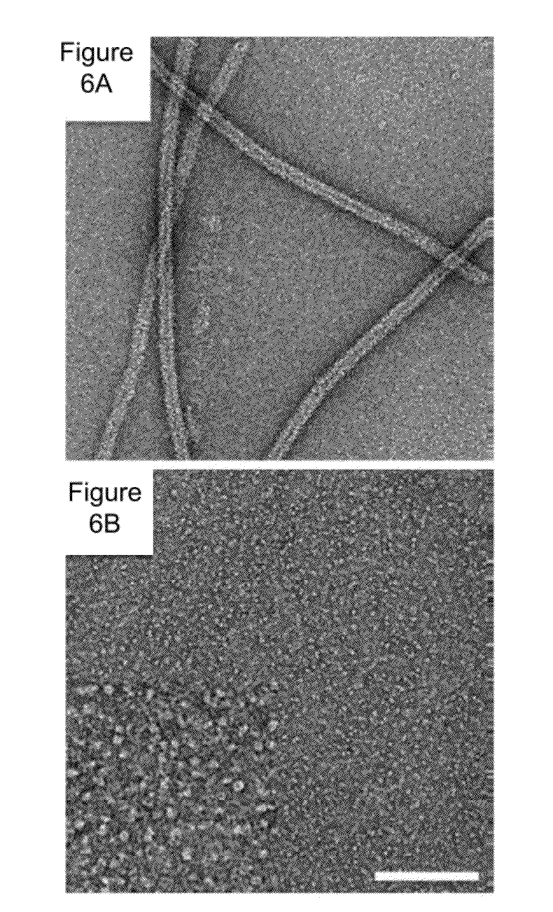

Rev in solution self-associates: monomers and dimers at low protein concentration associate to form high molecular weight filaments at higher concentrations (Wingfield et al., Biochemistry 30, 7527-34, 1991). The polymerization fits an isodesmic self-association model in which the association constant for the addition of a monomer to each aggregate is equal with a K_{d} of ˜1.0 μM (Cole et al., Biochemistry 32, 11769-75, 1993). The Rev filaments (FIG. 6a) are stable and are hollow with an outer diameter of ˜15 Å (Watts et al., J Struct Biol 121, 41-52, 1998). Addition of equimolar Fab to the filaments causes rapid depolymerization with the formation of small, uniformly sized complexes (FIG. 6b). In some views these complexes appear to have a central stain-penetrable hole (FIG. 6b, inset). In a similar manner the scFv also depolymerized Rev filaments. In both cases, the facile disruption of the protein-protein interactions is consistent with a high affinity interaction.

To approximately locate the Rev epitope, the C-terminal deletion mutant Rev^{Δ60-116} was used. Electron microscopy showed that this mutant by itself cannot assemble into a filament, probably due to the absence of the carboxy-terminal half. When the scFv was added, uniform complexes were formed that appeared similar, though smaller, to those observed with full-length Rev. This suggested that the epitope is present, and located in the N-terminal 1-59 residues of Rev.

Preparation and Characterization of Fab/Rev and scFv/Rev Complexes

Immune complexes were prepared by mixing the antibody fragments with a several-fold molar excess of Rev, then purifying them by means of metal chelate chromatography on Ni-Sepharose taking advantage of the C-terminal His tags. SDS-PAGE of the non-reduced Fab/Rev complex gave two main bands of ˜45 kDa and ˜16 kDa corresponding to oxidized

Fab and Rev (FIG. 3a, lane Fab/Rev). The predicted mass of Rev is 12,905 Da but it appears to have an anomalously low mobility in gel electrophoresis. Reduction of the complex also produced two bands, in this case Rev and an ˜25 kDa band corresponding to unresolved Fab heavy chain fragment (−24.5 kDa) and light chain (−23.5 kDa; FIG. 3b). Densitometry indicated a Fab/Rev 1:0.9 molar ratio, assuming equal Coomassie dye binding capacity. A similar analysis of the scFv/Rev complex under non-reducing (FIG. 3a, lane scFv/Rev) and reducing conditions (FIG. 3c) indicated a 1:0.87 molar ratio. Thus, both the Fab and scFv antibodies appear to form equimolar complexes with Rev.

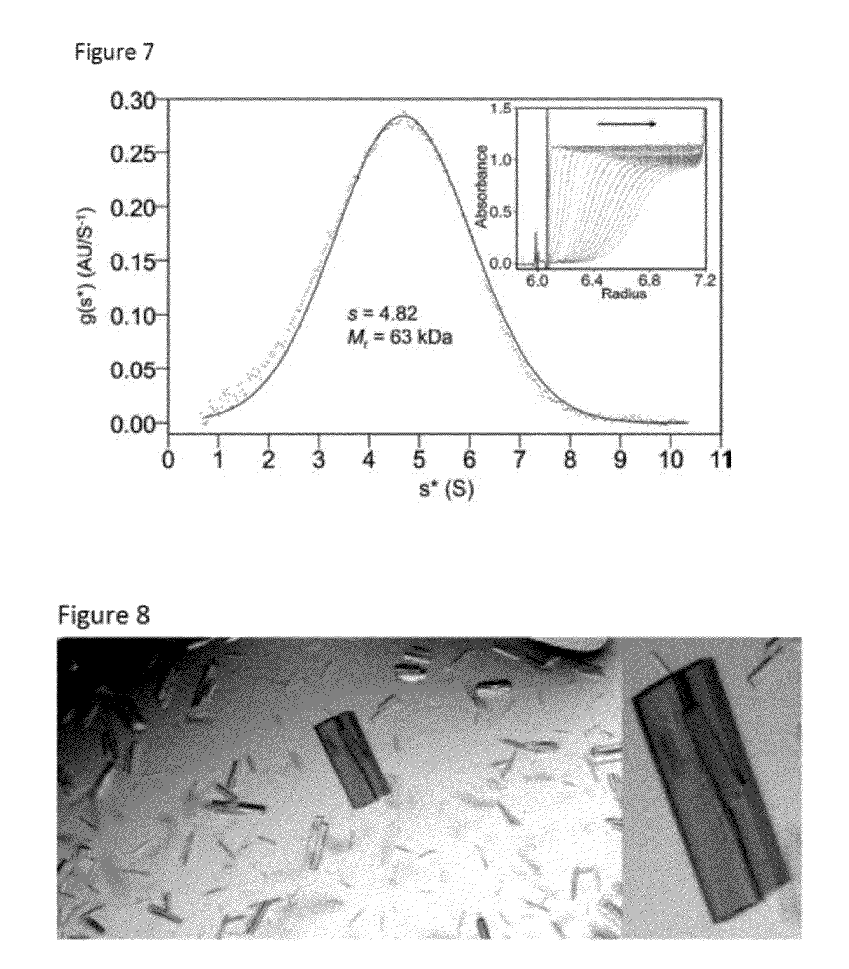

Following affinity purification, it is common to use gel filtration to finalize purification (polish), and at the same time confirm physical homogeneity as evidenced by symmetrical elution peaks. Unfortunately, both Rev and Rev complexed with antibody fragments adsorb strongly to the commonly used gel filtration matrices, ruling out this approach. As the primary aim of this work was to crystallize the immune complex it was important to ascertain monodispersity. For this purpose we used sedimentation velocity analysis after removing any protein aggregates by centrifugation at 100,000×g for 1-2 hours. Typical data (FIG. 7, insert) show a single moving boundary equivalent to a single species and characteristic of a monodisperse system. In a more detailed analysis, the sedimentation coefficient distribution plot (FIG. 7) also indicates a single species, analogous to a gel filtration peak (Philo et al., Anal Biochem 279, 151-63, 2000). From fitting the data, the diffusion coefficient can be obtained and, hence, a molecular weight estimate made ˜63 kDa (FIG. 7). This result is consistent with a complex of Rev and Fab in an equimolar ratio with a predicted mass of 60.81 kDa.

A more robust approach to mass and constituent stoichiometry determination is sedimentation equilibrium analysis. Protein gradients of Fab/Rev were analyzed assuming the system was ideal, for example, no reversible association, with a mass determination of 68 kDa (FIG. 4a). Small amounts of aggregate, evidenced as systematic error in residuals at the bottom of cell, accumulate during the course of the centrifugation. Variations in buffer composition and pH that might stabilize the complex during analysis have not yet been examined, though using lower temperatures had no effect. Even when the aggregated protein absorbance is truncated prior to data fitting, mass estimates are ˜65-68 kDa. If the gradient is analyzed as monomer-dimer system a better fit is obtained with a K_{d}˜0.1 mM, indicating a weak dimerization potential probably mediated by Rev (FIG. 4a). Native mass spectrometry also detects a Fab/Rev complex as the main component (with lesser amounts of oligomers of this 1:1 complex) but where none of the complexes contained dimeric Rev. The mass and stoichiometry determinations with the single chain antibody complex are clearer, the scFv/Rev having a determined mass of 40.4 kDa (FIG. 4b), which is close to that predicted for an equimolar complex (39.34 kDa).

### Crystallization of Fab/Rev Complex and Direct Identification of Epitope

Based on the physiochemical analysis, Fab/Rev appears homogeneous when freshly prepared, with the caveat that this assessment by analytical centrifugation was made using protein concentrations less than 1 mg/mL. In order to screen for homogeneity at higher protein concentrations, dynamic light scattering was used (FIG. 4d). This technique, which measures the translational diffusion coefficient, is widely used to assess the suitability of samples for crystallization (Borgstahl, Methods Mol Biol 363, 109-29, 2007). The Fab/Rev complex at concentrations between 1-9 mg/mL gave a main peak (−97% total protein) with mass estimations close to those obtained from centrifugation. (The mass determination by the DynaPro™ DLS plate reader is not as rigorous as that by sedimentation equilibrium but in this case was consistent). Aggregates did accumulate slowly when the complex was incubated at room temperature for up to 24 hours but these were easily removed by filtration or low speed centrifugation.

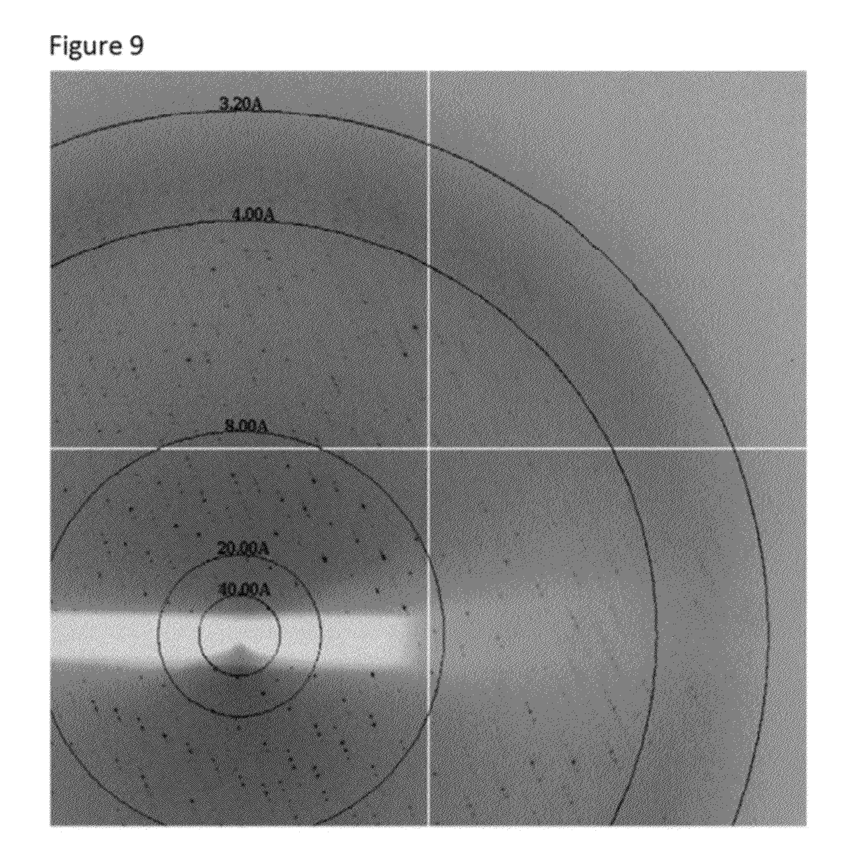

Fab/Rev was crystallized (see Material and Methods) forming long rods (FIG. 8). The crystals were suitable for X-ray structure determination and the diffraction pattern (FIG. 9) indicated resolution to beyond 3.3 Å. The data were indexed in a primitive triclinic crystal system, with unit-cell parameters a=87.7 Å, b=87.6 Å, c=177.4 Å, α=95.3°, β=94.9°, γ=104.3°. The structural determination of the Fab/Rev complex have been described elsewhere (DiMattia et al., Proc. Natl. Acad. Sci. U.S.A. 107, 5810-5814, 2010).

### HIV-1 Rev Epitope and Fab Paratope

The composition of the epitope was derived from the structure determination of the Fab/Rev complex at 3.2 Å resolution (DiMattia et al., Proc. Natl. Acad. Sci. U.S.A. 107, 5810-5814, 2010). Briefly, the epitope is conformational and located in the N-terminal region of Rev. Specific interactions with the Fab involve 16 residues encompassing amino acids within the N-terminal 63 residues that contact either one or both domains (V_{K} and V_{H}) of the Fab (FIG. 10; Table 1). The paratope (FIG. 10) reveals a Fab/Rev interface of ˜720 Å². This large binding footprint is consistent with high affinity binding and stabilization of the Fab/Rev 1:1 molar complex.

TABLE 1
Rev epitope residues engaged by the Fab SJS-R1

Buried Surface
Area (%)

| | Into heavy | Into light |
|---|---|---|
| Rev Residue | chain | chain |
| D11 | 20 | 30 |
| K14 | 59 | |
| A15 | 80 | 30 |
| L18 | 90 | 20 |
| 421 | 60 | |
| L22 | 80 | 20 |
| S25 | 10 | |
| N26 | 10 | |
| R48 | 40 | |
| Q51 | 40 | |
| I55 | 50 | 60 |
| R58 | 10 | 80 |
| L59 | 10 | 100 |
| S61 | | 10 |
| T62 | | 80 |
| Y63 | 10 | 60 |

### Discussion

### Stabilization of HIV-1 Rev for Structural Studies

The selection of Fab SJS-R1 is remarkable in that, although so far only this single clone has been obtained, it has such advantageous properties. The antibody binds to monomeric Rev with very high affinity producing a stable immune complex. At concentrations>80 μg/mL Rev rapidly polymerizes to form filaments. The Rev used for the rabbit immunizations was below this critical concentration and was therefore probably a monomer-dimer mixture. For selection of the chimeric rabbit/human Fab library that was generated from Rev-immunized rabbits, solid phase Rev immobilized via a C-terminal biotinylated Avitag was used. This modified Rev does not form filaments and although confirmation of its association state was not done, the immobilized protein at high dilution was likely to be either monomeric or dimeric and binding to these non-polymeric species was selected for. In future selections from this library, we will use Rev and mutant variants in other physical states in order to pan for clones binding to other epitopes. The high affinity of Fab SJS-R1, estimated to be ˜40 pM, is primarily due to an exceptionally low dissociation rate (FIG. 5). Because the panning protocol (Rader, Methods Mol. Biol. 525, 101-128, 2009) enriches clones with low dissociation rates it is conceivable that the Fab outcompeted other anti-Rev Fab early in the selection process. Mutant variants of Rev without the epitope of Fab SJS-R1 (Table 1) or epitope masking (Ditzel et al., J Immunol 154, 893-906, 1995) with purified Fab can be used to identify additional chimeric rabbit/human Fab clones.

Antibody fragment-mediated crystallization with high affinity reagents directed at conformation-sensitive epitopes is commonly used to improve crystallization by reducing protein flexibility and providing different surface contacts. This approach has been particularly important for membrane proteins (Hunte & Michel, Curr Opin Struct Biol 12, 503-8, 2002) but is also potentially useful for protein systems which exhibit physical heterogeneity due to self-association. Such systems may include proteins, as in the present study, that polymerize into filaments of indefinite length. Crystallization conditions may increase the propensity for self-association and antibody fragments which bind to an epitope located at or near protein-protein interaction interfaces may form stable, crystallizable complexes. The rapid depolymerization of Rev by the Fab or scFv clearly indicates binding to N-terminal protein oligomerization sites (FIG. 6) and indicates high affinity interactions. The Fab/Rev complex is monodisperse (FIG. 7) but during protracted sedimentation (>16 hours), some dimerization can occur. For this system, velocity sedimentation analysis (2 hours) appears to give a more reliable mass estimate. Nevertheless, the complex readily formed crystals (FIG. 8) which diffract to ˜3 Å (FIG. 9) and have been suitable for structural determination which will be reported elsewhere (DiMattia et al., Proc. Natl. Acad. Sci. U.S.A. 107, 5810-5814, 2010).

### Anti-HIV-1 Therapeutic Potential of Antibody Fragments

The high affinity and site of binding of Fab and scFv SJS-R1 predict that it may have anti-HIV-1 therapeutic potential. Although high affinity alone may not be predictive of efficacy, binding to the Rev epitope, which so effectively blocks Rev oligomerization, is significant. The function of Rev appears to depend on its ability to assemble as a multiprotein complex on the viral RNA targeting sequence RRE (see for example, Jain & Belasco, et al., Mol Cell 7, 603-14, 2001). Recent work suggests that following Rev binding to the RRE, it assembles into a tetramer and probably higher order complexes by oligomerization one Rev molecule at a time (Pond et al., Proc Natl Acad Sci USA 106, 1404-8, 2009). Direct visualization of the Rev-RRE by atomic force microscopy indicates a complex containing up to 13 Rev molecules (Pallesen et al., FEBS J 276, 4223-32, 2009). The binding affinities of the initial Rev-RRE interaction are 0.26 nM with subsequent bindings to tetramer in the range of 0.79-0.48 nM (Pond et al., Proc Natl Acad Sci USA 106, 1404-8, 2009). The Rev-Rev interactions on the RRE complex appear to be of much higher affinity than the simple self-association of Rev of 10 μM per monomer (Cole et al., Biochemistry 32, 11769-75, 1993).

Based on the work described herein, the affinity of both the initial Rev-RNA binding and the subsequent Rev oligomerization are weaker than the sub-nanomolar affinity of the Fab-Rev interaction (FIG. 5). The anti-HIV-1 potential of Fab and scFv SJS-R1 is, thus, based on its blocking the protein-protein interactions that are essential for Rev action. This could be achieved through anti-Rev intracellular immunization (Rondon & Marasco, Annu Rev Microbiol 51, 257-83, 1997) with scFv SJS-R1 targeted to the cytoplasm or nucleus. In a previous study, two murine-based anti-Rev scFv, which were targeted to the cytoplasm, inhibited HIV-1 replication in human T-cells and peripheral blood mononuclear cells (Wu et al., J Virol 70, 3290-3297, 1996). These antibodies had relatively low affinities (0.1-0.01 μM) and bound to epitopes in the C-terminal region of Rev (residues 69-94). Interestingly, Fab SJS-R1 binds to the N-terminal domain of Rev and the C-terminal domain (residues 69-116) is not observed in the Fab/Rev crystal structure being either non-structured or highly mobile (DiMattia et al., Proc. Natl. Acad. Sci. U.S.A. 107, 5810-5814, 2010). In another previous study, an anti-HIV-1 Rev nanobody was developed based on a llama heavy-chain only antibody, which inhibited HIV-1 replication and suppressed Rev-dependent expression of partially spliced and unspliced HIV-1 RNA (Vercruysse et al., J. Biol. Chem 285, 21768-21780, 2010; WO 2009/0147196).

Effective anti-HIV-1 agents based on anti-Rev intracellular antibodies may not be limited to any particular epitope but very high affinity binding would seem to be a prerequisite attribute. The anti-HIV-1 therapeutic potential of Fab SJS-R1 is not restricted to intracellular antibodies. The crystal structure of epitope and paratope of the Fab/Rev complex facilitates the computational modeling of lead peptides, peptidomimetics, or other small synthetic molecules that subsequently can be optimized by high-throughput screening of corresponding chemical libraries (Arkin & Wells, Nat Rev Drug Discov 3, 301-317, 2004). A small synthetic molecule that interferes with Rev action by mimicking Fab SJS-R1 would provide an anti-HIV-1 agent with potential oral bioavailability.

For the first time a Fab with rabbit variable domains and human constant domains derived by phage display has been produced against an HIV-1 protein. The antibody binds with very high affinity to a unique conformational epitope located in the N-terminal half of HIV-1 Rev. Both the Fab and its scFv derivative potently depolymerize Rev filaments indicating that their binding blocks a protein-protein interaction interface. The Fab/Rev complex is stable and readily forms crystals suitable for structural determination. This is also the first example of a chimeric/rabbit human Fab employed as a crystallization chaperone, promising broad utility for such agents in structural studies using X-ray diffraction. Based on the binding properties of the Fab and scFv described herein, and the central role that Rev plays in HIV viral infection, these molecules have therapeutic potential.

### Example 2

### Fusion Protein for Enhanced Intracellular Delivery

In some embodiments, the described anti-Rev antibody protein/peptide molecules are engineered or adapted so that they readily enter cells such as cells that are infected with HIV. This example describes a representative method for attaching a cell penetrating peptide (CPP; Fawell et al., Proc. Natl. Acad. Sci. 91, 664-668. 1994) to Rev scFv to direct cells to internalize the Rev scFv.

Using standard genetic engineering methods, a sequence encoding a CPP having the sequence RKKRRQRRR (SEQ ID NO: 11) was fused at the N terminus of the Rev scFv encoding sequence. The resultant Rev scFv-CPP fusion protein was purified and shown to bind Rev using methods described herein. Specifically, the interaction of the scFV-CPP with Rev was analyzed by negative-stain electron microscopy. scFv-CPP effectively depolymerized Rev filaments while in parallel controls without scFV-CPP the filaments remained intact.

### Example 3

### Ref scFv Intrabodies

In some embodiments, the described anti-Rev antibody protein/peptide molecules are expressed from nucleic acids that are delivered to target cell(s), thus providing the therapeutic protein/peptide directly in cells, such as cells that are infected with HIV. This example describes a representative method for generating a Rev scFv intrabody, to direct expression of Rev scFv intracellularly.

Using standard genetic engineering methods, a DNA sequence encoding Rev scFv is cloned into mammalian expression vector pcDNA3.1. Cells (that are normally susceptible to HIV-1 infection) are transformed with this Rev scFv vector and methods such as those described herein are used to determine whether that protects the cells from HIV-1 infection.

### Example 4

### HIV Antiviral Activity

This example provides representative non-limiting systems for testing and measuring the antiviral activity of a compound.

The anti-Rev based compounds disclosed are tested for their ability to inhibit HIV cell multiplication. The compounds are diluted into culture medium. MT-4, C8166, H9/III_{B} or other HIV-1-susceptible mammalian cells are grown at 37° C. in a 5% CO₂ atmosphere in RPMI 1640 medium, supplemented with 10% fetal calf serum (FCS), 100 IU/mL penicillin G and 100 μg/mL streptomycin. Cell cultures are checked periodically for the absence of mycoplasma contamination, e.g., with a MycoTect Kit (Gibco). Human immunodeficiency viruses type-1 is obtained from a culture collection or from another laboratory source, for instance, supernatants of persistently infected H9/III_{B}. Titration of HIV-1 is performed, for instance in C8166 cells by the standard limiting dilution method (dilution 1:2, four replica wells/dilution) in 96-well plates. The infectious virus titer is determined by light microscope scoring of cytopathicity after 4 days of incubation and the virus titers are expressed as CCID₅₀/mL.

Activity of the anti-Rev based compounds (e.g., SJS-R1 Fab or scFv, or another compound described herein) against HIV-1 multiplication in acutely infected cells in this representative assay is based on the inhibition of virus-induced cytopathicity in MT-4 cells. Briefly, 50 μL of culture medium containing 1×10⁴ cells are added to each well of flat-bottom microtiter trays containing 50 μL of culture medium with or without various concentrations of the test compounds. Then 20 μL of an HIV-1 suspension containing 100 CClD50 is added. After a 4-day incubation at 37° C., the number of viable cells was determined by the 3-(4,5-dimethylthiazol-1-yl)-2,5-diphenyltetrazolium bromide (MTT) method (Pauwels et al., J. Virol. Methods 1988, 20, 309-321) or any other art-recognized method. Cytotoxicity of the compounds may be evaluated in parallel with their antiviral activity, and for instance is based on the viability of mock-infected cells, as monitored by the MTT method. The 50% effective concentration value (EC₅₀) values for the test compounds are optionally calculated.

Alternatively, anti-viral activity can be examined for instance based on quantitation of an HIV protein, such as p24 levels, in HIV infected JC53-BL cells. Such quantitation can be determined using the Coulter HIV-1 p24 Antigen Neutralization Kit according to the manufacturer's recommendation. Briefly, two days (or some other period of time) post-treatment, JC53-BL cells are infected overnight with the X4-tropic HIV LAV (MOI=1). Subsequent to LAV infections, HIV p24 assays are performed by seeding 25,000 cells per well into 24-well plates, and p24 production is assayed from supernatants on day 3 post-infection using the HIV-1 p24 Antigen ELISA Test System (Beckman/Coulter/Immunotech, Brea, Calif.). Luciferase assays are performed in quadruplicate by measuring luciferase activity in detergent lysates one day post-infection with LAV (MOI=1) using the Steady-Glo Luciferase assay system (Promega, Madison, Wis.) and an EL 312e Microplate Bio-kinetics Reader (Bio-Tek Instruments, Winooski, Vt.). The luciferase assays permit quantitation of HIV infection of JC53-BL cells±treatment with a test agent such as SJS-R1 Fab, scFv, or another compound descried herein, whereas p24 assays measure secretion of infectious virus (and virus-like particles) into the supernatant.

The in vivo activity(s) (e.g., ability to prevent or decrease infection by HIV) of anti-Rev antibodies and fragments provided herein (including nucleic acids encoding such proteins and peptides) can also be assessed in animal models. For example, mouse HIV models are disclosed in Sutton et al. (Res. Initiat Treat. Action, 8:22-24, 2003) and Pincus et al. (AIDS Res. Hum. Retroviruses 19:901-908, 2003). Optionally, the therapeutic anti-Rev protein or peptide molecules in some embodiments include a peptide that assists in targeting of the therapeutic protein/peptide into the cytoplasm of target cells, for instance by facilitating membrane penetration or cross-membrane transport.

### Example 5

### Treatment of HIV Infection with SJS-R1-based Anti-Rev Molecules

This example describes exemplary, methods for treating HIV infection in a subject and exemplary methods for assessing efficacy of an antibody or antibody fragment that specifically binds HIV Rev for treating HIV infection or associated symptoms in a subject. However, one of skill in the art will appreciate that methods that deviate from these specific representative methods can also be used to treat HIV infection, AIDS, or an associated symptom caused by a lentivirus infection, in a subject.

Subjects known to be infected with HIV are selected. Subjects are treated daily with an anti-Rev antibody or binding fragment thereof (for example for 1, 2, 4, 8, 12, 18, 24, or more weeks), for example, the SJS-R1 Fab or SJS-R1 scFv or other anti-Rev antibody molecule as disclosed herein, or with a nucleic acid encoding a derivative anti-Rev antibody molecule such as an intrabody (for instance, in scFv format) or a transbody (in scFv, Fab, scFv-Fc, IgG1, or another format).

In an alternative embodiment, adoptive cell transfer is employed. Hematopoietic stem cells or T cells from an individual are manipulated ex vivo to express SJS-R1 scFv intrabody (or another SJS-R1-based anti-Rev molecule). The cells are expanded and then given back to the individual. As the intrabody renders these cells resistant to HIV-1 infection, they will persist in vivo. For a related strategy, see Perez et al. (Nat. Biotechnol. 26(70), 808-816, 2008).

Subjects are assessed for measures of HIV infection and/or AIDS progression (such as by monitoring the viral load and/or the level of CD4+ T-cells of a patient infected with the immunodeficiency virus), prior to initiation of therapy, periodically during the period of therapy, and/or at the end of the course of treatment. The viral load will decrease following administration of an effective agent.

The effectiveness of anti-Rev antibody therapy to treat or inhibit HIV infection in a subject can be demonstrated by an improvement in one or more measures of HIV infection (such as a 5%, 10%, 20%, 30% 50%, or 70% decrease in viral load, or increase in the level of CD4+ T cells) or a decrease in progression of one or more immunodeficiency virus infection associated symptoms, for example, compared to a control, such as an uninfected subject, a subject who was infected with HIV prior to treatment (for example, the same subject prior to treatment), or a subject infected with HIV but treated with placebo (e.g., vehicle only).

In additional treatment embodiments, other methods can be used to treat HIV in a human subject by administration of one or more Rev specific antibody molecules based on SJS-R1 Fab or scFv. Although particular methods, dosages, and modes of administrations are provided, one skilled in the art will appreciate that variations can be made without substantially affecting the treatment.

In particular examples, the subject is first screened to determine if they have HIV. Examples of methods that can be used to screen for HIV include a combination of measuring a subject's CD4+ T cell count and the level of HIV in serum blood levels. Additional methods using the Rev-specific antibody molecules described herein can also be used to screen for HIV.

In some examples, HIV testing consists of initial screening with an enzyme-linked immunosorbent assay (ELISA) to detect antibodies to HIV, such as to HIV-1. Specimens with a nonreactive result from the initial ELISA are considered HIV-negative unless new exposure to an infected partner or partner of unknown HIV status has occurred. Specimens with a reactive ELISA result are retested in duplicate. If the result of either duplicate test is reactive, the specimen is reported as repeatedly reactive and undergoes confirmatory testing with a more specific supplemental test (e.g., Western blot or an immunofluorescence assay (IFA)). Specimens that are repeatedly reactive by ELISA and positive by IFA or reactive by Western blot are considered HIV-positive and indicative of HIV infection. Specimens that are repeatedly ELISA-reactive occasionally provide an indeterminate Western blot result, which may be either an incomplete antibody response to HIV in an infected person, or nonspecific reactions in an uninfected person. IFA can be used to confirm infection in these ambiguous cases. In some instances, a second specimen will be collected more than a month later and retested for subjects with indeterminate Western blot results. In additional examples, nucleic acid testing (e.g., viral RNA or proviral DNA amplification method) can also help diagnosis in certain situations.

The detection of HIV in a subject's blood is indicative that the subject has HIV and is a candidate for receiving the therapeutic compositions disclosed herein. Moreover, detection of a CD4+ T cell count below 350 per microliter, such as 200 cells per microliter, is also indicative that the subject is likely to have HIV.

Pre-screening is not required prior to administration of the therapeutic compositions disclosed herein

In particular examples, the subject is treated prior to administration of a therapeutic agent that includes one or more antiretroviral therapies known to those of skill in the art. However, such pre-treatment is not always required, and can be determined by a skilled clinician.

Following subject selection, a therapeutically effective dose of an anti-Rev antibody molecule described herein is administered to the subject (such as an adult human or a newborn infant either at risk for contracting HIV or known to be infected with HIV). Additional agents, such as anti-viral agents, can also be administered to the subject simultaneously or prior to or following administration of the disclosed agents. Administration can be achieved by any method known in the art, such as oral administration, inhalation, intravenous, intramuscular, intraperitoneal, or subcutaneous. Optionally, the therapeutic anti-Rev protein or peptide molecules include a peptide that assists in targeting of the therapeutic protein/peptide into the cytoplasm of target cells, for instance by facilitating membrane penetration or cross-membrane transport.

The amount of the composition administered to prevent, reduce, inhibit, and/or treat HIV or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., HIV) in a subject without causing a substantial cytotoxic effect in the subject. An effective amount can be readily determined by one skilled in the art, for example using routine trials establishing dose response curves. As such, these compositions may be formulated with an inert diluent or with a pharmaceutically acceptable carrier.

In one specific example, antibodies are administered at 5 mg per kg every two weeks or 10 mg per kg every two weeks depending upon the particular stage of HIV. In an example, the antibodies are administered continuously. In another example, antibodies or antibody fragments are administered at 50 μg per kg given twice a week for 2 to 3 weeks.

Administration of the therapeutic compositions can be taken long term (for example over a period of months or years).

Following the administration of one or more therapies, subjects having HIV can be monitored for reductions in HIV levels, increases in a subjects CD4+ T cell count, or reductions in one or more clinical symptoms associated with HIV. In particular examples, subjects are analyzed one or more times, starting seven days following treatment. Subjects can be monitored using any method known in the art. For example, biological samples from the subject, including blood, can be obtained and alterations in HIV or CD4+ T cell levels evaluated.

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50%, or at least 70% in HIV infection, HIV replication or combination thereof. A partial response may also be an increase in CD4+ T cell count such as at least 350 T cells per microliter.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An anti-Rev antibody or a fragment thereof which maintains binding activity to HIV-1 Rev, comprising:
a V_{H} region with a framework and comprising:
a first CDR comprising the amino acid sequence GFWLNW (positions 31-36 of SEQ ID NO: 2);
a second CDR comprising the amino acid sequence AIYRGSGSEWYASWAKG (positions 50-66 of SEQ ID NO: 2); and
a third CDR comprising the amino acid sequence AADTTDNGYFTI (positions 95-106 of SEQ ID NO: 2); or
a V_{H} region having a sequence at least 90% identical to SEQ ID NO: 2; and
a V_{L} region with a framework and comprising:
a first CDR comprising the amino acid sequence QASQSISSWLS (positions 25-35 of SEQ ID NO: 4);
a second CDR comprising the amino acid sequence DASNLAS (positions 51-57 of SEQ ID NO: 4); and
a third CDR sequence comprising the amino acid sequence LGGYPAASYRTA (positions 90-101 of SEQ ID NO: 4); or
a V_{L} region having a sequence at least 90% identical to SEQ ID NO: 4.

2. The anti-Rev antibody or fragment thereof of claim 1, wherein the V_{L} region is a Vκ region.

3. The anti-Rev antibody or fragment thereof of claim 2, wherein the framework of the V_{H} region is at least 90% or more identical to the framework of SEQ ID NO: 2, and the framework of the Vκ region is at least 90% or more identical to the framework of SEQ ID NO: 4.

4. The anti-Rev antibody or fragment thereof of claim 3, wherein the framework of the V_{H} region is 95% or more identical to the framework of SEQ ID NO: 2, and the framework of the Vκ region is at least 95% or more identical to the framework of SEQ ID NO: 4.

5. The anti-Rev antibody or fragment thereof of claim 1, wherein the V_{H} region comprises the sequence shown in positions 1-117 of SEQ ID NO: 2, and the Vκ region comprises the sequence shown in positions 2-111 of SEQ ID NO: 4.

6. The anti-Rev antibody or fragment thereof of claim 1, wherein the fragment thereof which maintains binding activity to Rev is an Fab fragment, an (Fab′)₂, an Fv fragment, an single chain Fv fragment (scFv), an scFv-Fc, an intrabody, an IgG, another bivalent antibody format or transbody.

7. The anti-Rev antibody or fragment or variant thereof of claim 1, which is humanized.

8. The anti-Rev Fab fragment of claim 6, which is Fab SJS-R1.

9. The anti-Rev Fab fragment of claim 6, which is scFv SJS-R1.

10. An isolated antibody or antibody fragment that binds the same epitope site as does the antibody or fragment of claim 1.

11. A pharmaceutical composition comprising the anti-Rev antibody or fragment thereof of claim 1.

12. The pharmaceutical composition of claim 11, further comprising another therapeutic agent.

13. An isolated polynucleotide encoding the V_{H} or V_{L} region of the anti-Rev antibody or fragment thereof of claim 1.

14. The isolated polynucleotide of claim 13, comprising the sequence shown in SEQ ID NO: 1, SEQ ID NO: 3, or both.

15. A vector comprising the isolated polynucleotide of claim 13.

16. An isolated recombinant host cell expressing the polynucleotide of claim 13, wherein the cell is a prokaryotic cell or an immortalized eukaryotic cell line.

17. The anti-Rev antibody or fragment thereof of claim 1, which is labeled with one or more of a radionuclide, fluorophore, coloring, enzyme, enzymatic substrate, enzymatic factor, enzymatic inhibitor or ligand.

18. A method comprising contacting Rev protein with the antibody or antibody fragment of claim 1.

19. The method of claim 18, which is a method of inhibiting or preventing or reversing multimerization/polymerization of Rev.

20. The method of claim 18, which is a method for preventing or inhibiting replication of a lentivirus.

21. The method of claim 18, which is a method of reducing infectivity or replication of a lentivirus.

22. The method of claim 18, which is a method of inhibiting Rev function in a cell infected with a lentivirus.

23. The method of claim 18, which takes place in a cell.

24. The method of claim 23, wherein the cell is a mammalian cell infected with a lentivirus.

25. The method of claim 18, wherein the Rev protein is from a lentivirus selected from the group consisting of HIV-1, HIV-2, SIV, FIV and other lentiviruses that expresses Rev.

26. The method of claim 25, wherein the lentivirus is a human lentivirus.

25. A method of treating a disease or symptom associated with Rev expression or activity in an animal, comprising administering to an animal with said disease or symptom a therapeutically effective amount of the anti-Rev antibody or fragment of claim 1, thereby treating the disease or symptom.

26. The method of claim 25, wherein the subject is infected with a lentivirus.

27. The method of claim 26, wherein the lentivirus is HIV-1, HIV-2, SIV, FIV or another lentivirus that expresses Rev.

28. The method of claim 26, wherein the lentivirus is a human lentivirus.

29. A peptide or small molecule that binds to Rev at the same epitope site as does SJS-R1 Fab, and which inhibits, prevents or reverses Rev multimerization/polymerization.
